(19) United States
(12) Patent Application Publication
(10) Pub. No.: US 2021/0231670 A1
(43) Pub. Date: Jul. 29, 2021

(54) METHODS OF EVALUATING TREATMENT OUTCOME IN HIGH GRADE SEROUS OVARIAN CANCER

(71) Applicant: THE REGENTS OF THE UNIVERSITY OF COLORADO, A BODY CORPORATE, Denver, CO (US)

(72) Inventors: Benjamin G. Bitler, Denver, CO (US); Lindsay J. Wheeler, Denver, CO (US)

(21) Appl. No.: 17/251,645

(22) PCT Filed: Jun. 14, 2019
(86) PCT No.: PCT/US2019/037289

§ 371 Date: Dec. 11, 2020

Related U.S. Application Data
(60) Provisional application No. 62/685,107, filed on Jun. 14, 2018.

(51) Int. Cl. G01N 33/574; A61K 45/06; C12N 15/113; A61K 33/243; G01N 33/68

(52) U.S. Cl. CPC G01N 33/57449; A61K 45/06; C12N 15/113; C12N 2310/531; G01N 33/6848; G01N 2333/4704; A61K 33/243

(57) ABSTRACT

Disclosed herein are method and compounds useful in the analysis, diagnosis, and treatment of high grade serous ovarian carcinoma (HGSOC). Also disclosed are methods, compounds, and compositions useful in regulating Chromobox 2 (CBX2) expression and therapies for stem-ness, anoikis escape, HGSOC dissemination, and HGSCO chemoresistance. Applicants have identified CBX2 expression as being significantly elevated in HGSOC cells and tissues compared to benign counterparts. Also disclosed is elevated CBX2 expression in HGSOC cell lines, as well as elevated CBX2 expression in cells that are forced to grown in suspension. Reducing CBX2 results in inhibition of anchorage-independent proliferation and potentiation of anoikis-dependent apoptosis, as well as re-sensitization of HGSOC cells to platinum-based chemotherapy.

### CROSS-REFERENCE TO RELATED APPLICATIONS

This application claims benefit of priority pursuant to 35 U.S.C. § 119(e) of U.S. provisional patent application No. 62/685,107 entitled “Methods of Evaluating Treatment Outcome in High Grade Serous Ovarian Cancer,” filed on 14 Jun. 2018, which is hereby incorporated by reference in its entirety.

### GOVERNMENT LICENSE RIGHTS

This invention was made with government support under grant number CA194318 awarded by the National Institutes of Health (NIH). The government has certain rights in the invention.

### FIELD

The disclosed processes, methods, and compound are directed to diagnosing and treating conditions related to elevated CBX2 expression, especially in high-grade serous ovarian carcinoma cells and tissues.

### BACKGROUND

Epithelial ovarian cancer is the deadliest gynecologic malignancy and annually accounts for over 220,000 deaths worldwide. In the US, over 22,000 new cases of ovarian cancer are diagnosed each year and over 14,000 women succumb to the disease. The majority of these cases are classified as high-grade serous ovarian carcinoma (HGSOC). HGSOC tends to be diagnosed at a late stage, when cancer has already spread beyond the pelvis, and will recur in the majority of cases. Current evidence suggests that HGSOC originates from transformed secretory fallopian tube epithelium (FTE) cells located on the fimbriated end of the fallopian tube. Precursor lesions, defined by TP53 mutations, include serous tubal intraepithelial carcinoma (STIC), which is focal and displays a cytologic appearance similar to HGSOC. Cells within STIC lesions demonstrate anoikis resistance or anchorage-independent cell survival by exfoliation from the fallopian tube-associated extracellular matrix and dissemination to the ovary and/or peritoneum. Ovarian, fallopian, and primary peritoneal carcinomas differ from other epithelial cancers that metastasize to distant sites predominantly via the circulatory or lymphatic systems (e.g., breast, endometrial) by spreading directly to the ovaries and the abdominal cavity independent of the lymphatic or vascular system. As HGSOC cells spread to the abdominal cavity they promote the production of ascites, a collection of intra-peritoneal fluid containing immune cells, tumor cells, and cytokines, along with other cellular and acellular factors. Notably, the prevalence of ascites is directly correlated to disease stage. For instance, 89% of stage III/IV patients present with some degree of ascites. Tumor cells within ascites are hypothesized to be a subpopulation of cells that contribute to disseminated, recurrent, and chemoresistant disease. However, the genetic drivers of HGSOC dissemination and anchorage-independent survival remain unclear.

A significant proportion of “stem”-like cells have been detected in the ascites fluid associated with HGSOC. One group of transcriptional repressors, the polycomb group (PcG) of proteins, are candidates for producing and maintaining this “sternness” as they have been shown to inhibit cellular differentiation and maintain a stem-like transcriptional program. PcG proteins assemble in two main Polycomb repressive complexes, PRC1 and PRC2. PRC1 and 2 epigenetically repress pro-differentiation and tumor suppressor genes, and are important in several cancer types including prostate, breast, and HGSOC. Epigenetic “readers”, known as chromobox (CBX) proteins, play a critical role in PRC1 repressive activity by recognizing methylated histones through their chromobox domain. In 2014, Clermont et al. initially identified an oncogenic role for CBX2 through a genotranscriptomic meta-analysis in human cancers. In breast and prostate cancers, they reported that CBX2 upregulation and amplification significantly correlated with metastatic progression and lower overall survival. CBX2 depletion reduced cell viability and promoted apoptosis in metastatic prostate cancer, suggesting that CBX2 drives key regulators of cell proliferation and metastasis. Gui et al. evaluated the role of 12 PcG proteins in primary and recurrent ovarian cancer and found that immunohistochemistry (IHC) demonstrated significantly higher levels of CBX2 expression in recurrent tumors compared to primary tumors at presentation (primary ovarian tissue at presentation n=100, recurrent disease at relapse n=50, p<0.001). However, the role of CBX2 in HGSOC progression is unknown.

### SUMMARY

Disclosed herein are methods of assessing chemoresistance of high grade serous ovarian carcinoma (HGSOC) in a patient suffering from the same, the method comprising the analyzing for Chromobox 2 (CBX2) levels in a patient's biological sample, wherein analyzing comprises analyzing at least one HGSOC cell, and wherein, if the CBX2 levels in the patient's at least one HGSOC cell are higher than in a control sample, the patient's HGSOC is resistant to chemotherapy. In some embodiments, the chemotherapy may comprise cisplatin, and the patient with high CBX2 levels may be further counseled not to receive cisplatin as a HGSOC treatment. Also disclosed are methods of treating or preventing HGSOC in a patient, comprising administering to the patient a therapeutically effective of a compound that inhibits and/or downregulates CBX2, for example by a compound comprising an antibody, siRNA, ribozyme, antisense, aptamer, peptidomimetic, small molecule, or any combinations thereof.

Also disclosed are methods of determining a treatment for a patient with, or at risk of developing a cancer, the method comprising obtaining a first sample from the patient comprising one or more cancerous or pre-cancerous cells, obtaining a second sample from the patient that is similar to the first sample but does not comprise cancerous or pre-cancerous cells, processing the first sample and the second sample to analyze at least one biomarker related to CBX2, quantifying the amount of biomarker in the first sample and the second sample, wherein if the amount of CBX2-related biomarker in the first sample is greater than the amount of CBX2-related biomarker in the second sample, the patient is identified as having aggressive or chemoresistant cancer, wherein the first sample is derived from ovarian, uterine, or fallopian tissue. In some cases, the first sample may include one or more HGSOC cells, the biomarker may comprise CBX2 protein or fragment thereof, or at least on nucleic acid, such as an mRNA sequence from the CBX2 gene, and the amount of biomarker may be quantified by immunoblot or mass spectrometry. In some cases, a second biomarker may also be obtained from each sample, wherein the second biomarker is not CBX2. In many embodiments, chemoresistance may be to a platinum-based chemotherapeutic agent, such as cisplantin, carboplatin, or oxaplatin.

Also disclosed herein are methods of determining whether a tumor is likely to metastasize. The method comprising steps of measuring expression levels of at least one CBX2-associated biomarker from a sample of the tumor tissue, measuring expression levels of at least one CBX2-associated biomarker from a sample of non-tumor tissue, wherein if the expression level of the at least one CBX2-associated biomarker from the tumor sample is greater than the expression level of the at least one CBX2-associated biomarker from the non-tumor sample, the tumor is determined to be likely to metastasize. In many cases, the first sample is derived from tissue of Müllerian origin, and the first and second samples may be derived from ovarian or fallopian tissue. In some cases, the first sample may include one or more HGSOC cells, the biomarker may comprise CBX2 protein or fragment thereof, or at least on nucleic acid, such as an mRNA sequence from the CBX2 gene, and the amount of biomarker may be quantified by immunoblot or mass spectrometry. In some cases, a second biomarker may also be obtained from each sample, wherein the second biomarker is not CBX2.

Kits for diagnosing a chemoresistant cancer in a patient are also disclosed herein. IN many embodiments the kits comprise: a quantitation reagent comprising one or more detectors specific for at least one CBX2-associated biomarker from at least one biological sample; a detection reagent; instructions for using the kit to diagnose a patient as having ovarian cancer when the expression levels of the CBX2-associated biomarker in the biological sample from the patient is higher than the expression level of the same biomarkers in a control subject or control biological sample. In many cases, the first sample is derived from tissue of Müllerian origin, and the first and second samples may be derived from ovarian or fallopian tissue. In some cases, the first sample may include one or more HGSOC cells, the biomarker may comprise CBX2 protein or fragment thereof, or at least on nucleic acid, such as an mRNA sequence from the CBX2 gene, and the amount of biomarker may be quantified by immunoblot or mass spectrometry. In some cases, a second biomarker may also be obtained from each sample, wherein the second biomarker is not CBX2.

Methods of inhibiting or reducing proliferation in a cancer cell are also disclosed herein. The methods may comprise steps of: contacting the cancer cell with a compound or molecule that inhibits CBX2 expression; allowing the compound to reduce the amount of CBX2 protein in the cell; and thereby reducing or inhibiting proliferation of the cell compared to a control cell that is not contacted with the compound. In many cases, these methods may be practiced on cells in-vitro, or in-vivo, and the cells may be mammalian cells, such as human cells. In most embodiments, the compound may be a nucleic acid, such as a short hairpin ribonucleic acid, or the compound may comprise two or more amino acids. The methods may further comprise as a step of contacting the cell with one or more chemotherapeutic agents which may be performed before the reducing step.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows CBX2 is overexpressed in high grade serous carcinoma and portends poor prognosis.

FIG. 2 shows inhibition of CBX2 impairs HGSOC cell proliferation.

FIG. 3 shows suspension growth and CBX2 modulation in multiple cell lines.

FIG. 4 shows CBX2 expressed in advanced HGSOC.

FIG. 5 shows CBX2 antibody validation for immunohistochemistry

FIG. 6 shows loss of CBX2 sensitizes HGSOC to chemotherapy.

FIG. 7 shows CBX2 knockdown sensitizes OVCAR8 and PEO1 cells to cisplatin.

FIG. 8 shows a correlation of CBX2 expression and autophagy, apoptosis, and EMT.

FIG. 9 shows inhibition of CBX2 decreases stemness.

FIG. 10 shows CBX2 knockdown leads to loss of ALDH3A1 expression.

### DETAILED DESCRIPTION

Disclosed herein are studies showing that CBX2 is overexpressed in primary HGSOC tumors and that CBX2 protein is upregulated in HGSOC cells grown in an anchorage-independent fashion (forced suspension). In a primary human HGSOC tumor microarray high CBX2 expression was observed in a majority of specimens. Also shown is that the loss of CBX2 inhibits proliferation, reduces stemness, and increases cisplatin sensitivity. Also disclosed are methods of diagnosing advanced cases of HGSOC, HGSOC that may be resistant to one or more anti-cancer therapies, for example platinum-based chemotherapy, such as cisplatin therapy. Also disclosed are compounds and methods for treating a subject having advanced or chemoresistant HGSOC, wherein the compounds and methods reduce CBX2 expression in HGSOC cells and/or reduce dissemination of primary tumors.

High grade serous ovarian carcinoma (HGSOC) is often diagnosed at an advanced stage. Chromobox 2 (CBX2), a polycomb repressor complex subunit, plays an oncogenic role in other cancers, but little is known about its role in HGSOC. Applicants hypothesized that CBX2 upregulation promotes HGSOC via induction of a stem-like transcriptional profile and inhibition of anoikis. Examination of Gene Expression Omnibus (GEO) datasets and The Cancer Genome Atlas (TCGA) established that increased CBX2 expression conveyed chemoresistance and worse disease-free and overall survival. In primary HGSOC tumors, Applicants observed CBX2 expression was significantly elevated compared to benign counterparts. In HGSOC cell lines, forced suspension promoted CBX2 expression. Subsequently, CBX2 knockdown inhibited anchorage-independent proliferation and potentiated anoikis-dependent apoptosis. Furthermore, CBX2 knockdown re-sensitized cells to platinum-based chemotherapy. Forced suspension promoted increased ALDH activity and ALDH3A1 expression and CBX2 knockdown led to a decrease in both ALDH activity and ALDH3A1 expression. Investigation of CBX2 expression on a HGSOC tissue microarray revealed CBX2 expression was apparent in both primary and metastatic tissues. CBX2 is an important regulator of stem-ness, anoikis escape, HGSOC dissemination, and chemoresistance and potentially serves as a novel therapeutic target.

### Discussion

Applicants have discovered that CBX2 is upregulated in HGSOC, high CBX2 expression portends poorer survival, and increased CBX2 expression correlates with platinum resistance. Applicants have also discovered that CBX2 is overexpressed in HGSOC primary tumors, as well as in cell lines that have escaped anoikis in suspension culture. Here, Applicants demonstrate that the loss of CBX2 is associated with decreased proliferation of HGSOC cells in multiple culture conditions, an increase in chemotherapy sensitivity, and a reduction in stem-like cells. Lastly, utilizing a HGSOC tissue microarray of advanced stage primary patient tumors Applicants found CBX2 protein expression was expressed in a majority of tumors.

CBX2 was found to be highly expressed in primary HGSOC tumors from seven patients. Clinically, three of these patients had more extensive peritoneal disease (Table 2) suggesting that CBX2 could serve as a predictive marker of advanced disease, reinforcing the potential clinical significance of CBX2. Moreover, examination of five patients with matched primary tumor, ascites-associated tumor cells, and distant metastasis revealed three of the five patients had an increase in CBX2 expression in distant metastasis/ascites-associated tumor cells compared to primary tumors. This highlights that CBX2 is potentially important in driving HGSOC progression, however there are indeed other contributing factors. Notably we observed that high expression of CBX2 correlated to a loss of an active tumor suppressor, FOXO3. This suggests that the downregulation of FOXO3 independent of CBX2 could drive tumor progression. As we have attempted to elucidate the mechanism behind CBX2-dependent activity we see common themes of increased proliferation and stem-like differentiation, which appeared to lead to a more aggressive and chemoresistant phenotype. The loss of CBX2 led to the loss of stemness measured through ALDH activity, ALDH3A1 expression, and SOX4 expression suggesting that reduced stemness is a major driver of these phenotypes. In addition, we linked the anoikis-induced autophagy, EMT, and apoptosis response to CBX2 expression. Further investigations will functionally evaluate the relationship between CBX2 and these key survival processes.

TABLE 2
Clinical characteristics of patient-derived protein lysates

| Label | Specimen | Pathologic Diagnosis | Indication for surgery | Clinical diagnosis |
|---|---|---|---|---|
| FTE | Normal | HGSC, right fallopian | Left adnexal mass, left | Stage IIIC high grade |
| (952) | fallopian | tube not involved | hydroureter and | serous ovarian |
| | tube | | hydronephrosis | cancer, recurrent |
| FTE | Normal | Endometrioid, | Endometrial | Stage IA |
| (968) | fallopian | endometrial | adenocarcinoma | Endometrioid, |
| | tube | adenocarcinoma, FIGO | diagnosed on biopsy | endometrial |
| | | grade 1 | | adenocarcinoma, |
| | | | | FIGO Grade I |
| FTE | Normal | Lipoleiomyomata, | Incidentally found 20 cm | Benign pelvic mass |
| (1008) | fallopian | bilateral fallopian tubes | right adnexal mass on CT | |
| | tube | without histopathologic | scan | |
| | | abnormality | | |
| Benign | Normal | Uterus, cervix, and | Malignant cells on pap | Negative for |
| (982) | ovary | bilateral ovaries and | smear | malignancy. |
| | | fallopian tubes negative | | |
| | | for malignancy | | |
| HGSC | Omentum | HGSC | Inflamed gallbladder, | Stage IVB high grade |
| (905) | | | omental cake, and | serous ovarian |
| | | | adnexal masses | carcinoma, recurrent |
| | | | suspicious for ovarian | |
| | | | cancer | |
| HGSC | Tumor/ | HGSC | Adnexal masses, | Stage IVB high grade |
| (930) | ovary | | concerning for ovarian | serous ovarian |
| | | | cancer | cancer, recurrent |
| HGSC | Tumor/ | HGSC | Pelvic mass, ascites, and | Stage IIIC high grade |
| (945) | omentum/ | | carcinomatosis | serous ovarian |
| | ovary | | concerning for ovarian | cancer, recurrent |
| | | | cancer | |
| HGSC* | Tumor/ | HGSC | Abdominal distension, | Stage IIIC high grade |
| (966) | ovary | | bilateral adnexal masses | serous ovarian |
| | | | | cancer, recurrent |
| HGSC | Tumor/ | HGSC | Omental caking and | Stage IIIC high grade |
| (995) | ovary | | adnexa masses | serous ovarian cancer |
| | | | suspicious for ovarian | |
| | | | cancer. | |
| HGSC | Tumor/ | HGSC | Left adnexal mass, left | Stage IIIC high grade |
| (952) | ovary | | hydroureter and | serous ovarian |
| | | | hydronephrosis | cancer, recurrent |
| HGSC | Tumor/ | HGSC | Bilateral adnexal masses | Stage IIIC high grade |
| (1003) | ovary | | | serous carcinoma of |
| | | | | the fallopian tube |
| Other | Tumor/ | Low grade serous | Flank pain, adnexal | Stage IV low grade |
| histotype | omentum | carcinoma | mass | serous ovarian |
| (953) | | | | cancer, recurrent |
| Other | Tumor/ | Carcinosarcoma | Pelvic mass in setting of | Stage IVB |
| histotype | ovary | | carcinoma of unknown | carcinosarcoma of the |
| (950) | | | origin | ovary |
| Other | Tumor/ | Carcinosarcoma | Small bowel obstruction, | Stage IIIC ovarian |
| histotype | ovary | | new diagnosis | carcinosarcoma |
| (991) | | | carcinosarcoma | |
| Other | Tumor/ | Mucinous | Ascites, bloating, cytology | Stage IIIC mucinous |
| histotype | ovary | adenocarcinoma, | positive for | adenocarcinoma of |
| (993) | | intestinal type, primary | adenocarcinoma | the ovary |
| | | tumor site: right ovary | | |
| HGSC* | Tumor/ | HGSC | Abdominal | Stage IIIC high grade |
| (966) | ovary | | distension, | serous ovarian |
| | | | bilateral adnexal masses | cancer, recurrent |

| | | Surgical | CBX2 |
|---|---|---|---|
| Label | Surgical findings (when relevant) | outcome | status |
| FTE | Bilateral adnexal masses 4 × 5 cm on the right and | Optimal | Faint |
| (952) | 8 × 10 cm on the left. Significant retroperitoneal tumor | debulking to | |
| | burden, left greater than right necessitating | <1 cm residual | |
| | aggressive dissection and ligation of internal iliac | disease | |
| | artery to remove tumor. | | |
| | Per pathology report: right fallopian tube NOT | | |
| | INVOLVED with HGSOC | | |
| FTE | Mildly enlarged uterus, normal-appearing right | Not applicable. | Faint |
| (968) | fallopian tube and ovary. Previous surgically absent | | |
| | left fallopian tube and ovary. There was no disease | | |
| | noted in the pelvis. | | |
| FTE | Enlarged, approximately 20 × 20 cm retroperitoneal | Optimal | Faint |
| (1008) | mass arising within the broad ligament attached to the | debulking | |
| | uterus. Both ovaries and fallopian tubes were normal | | |
| | bilaterally. Extensive retroperitoneal fibrosis, | | |
| | necessitating complete ureterolysis. | | |
| Benign | Uterus slightly enlarged and globular. Significant intra- | Not applicable | Faint |
| (982) | abdominal adhesions. *No specific mention of ovaries | | |
| | in findings | | |
| HGSC | Extensive approximately 30 × 20 × 10 cm omental | Optimal | Bright |
| (905) | cake incorporating the infracolic and gastrocolic | debulking to <1 cm, | |
| | omentum, removed completely. Splenic hilar tumor | however residual consisted | |
| | and distal pancreatic surface tumor, removed | of thousands of less | |
| | completely with splenectomy and distal | than 1 cm diffuse peritoneal and | |
| | pancreatectomy. Diffuse intraabdominal mesenteric | mesenteric nodules, as well | |
| | and peritoneal masses up to 4 cm in size, debulked | as areas of plaque which had | |
| | with the use of argon beam to less than 1 cm | been debulked to | |
| | thickness of the removed plaques. Right adnexal | less than 1 cm thickness, but | |
| | mass approximately 8 cm. Diffuse peritoneal disease | nonetheless consisted of | |
| | measuring in the thousands, all debulked to less than | plaque-like disease. | |
| | 1 cm, discrete lesions or plaque-like in thickness | | |
| | areas. Gallbladder removed with thickened wall of the | | |
| | gallbladder, but no intrinsic luminal tumor. | | |
| HGSC | Bilateral adnexal masses, left with surface | Poor candidate for primary | Bright |
| (930) | involvement with a tumor process, approximately 10 | debulking given extensive right | |
| | cm in size. The right approximately 15 cm in size and | diaphragmatic disease. | |
| | mostly cystic. Small volume intraabdominal ascites, | Underwent optimal | |
| | approximately 50 cc. The right hemidiaphragm | cytoreduction to | |
| | infiltration approximately 3 to 4 cm thick and across an | R0 after 3 cycles | |
| | approximately 10 cm area making it difficult to remove | of neoadjuvant | |
| | without complete diaphragmatic resection. No | chemotherapy | |
| | obviously thickened omentum on laparoscopic | | |
| | assessment. Multiple small nodules ranging from 5 to | | |
| | 10 mm diffusely through the peritoneum. | | |
| HGSC | Approximately 2 L of ascites. Omental disease with | Optimal | Moderate |
| (945) | numerous peritoneal-based lesions, the majority less | debulking to | |
| | than 1 cm and those greater than 1 cm debulked to | <1 cm, however | |
| | less than 1 cm residual volume. Tumor roll in the | innumerable miliary disease | |
| | omentum approximately 15 cm × 9 cm and 4 cm, | on the bilateral | |
| | debulked to less than 1 cm residual. Bilateral adnexal | diaphragms and peritoneal | |
| | masses, approximately 10 cm on the left and | surfaces | |
| | approximately 4 cm on the right, densely adherent in | | |
| | the pelvis, with bilateral ureterolysis secondary to | | |
| | retroperitoneal fibrosis allowing resection of the | | |
| | ovaries. Anterior roll of tumor in the bladder | | |
| | peritoneum approximately 2 × 4 × 2 cm, completely | | |
| | removed. Tumor along the cecum and appendix | | |
| | resulting in removal of what appeared to be appendix | | |
| | involved with tumor, approximately 2 cm × 2 cm × 4 | | |
| | cm. Tumor mass in the lesser curvature of the | | |
| | stomach approximately 2 × 4 × 2 cm, removed | | |
| | completely. At the completion of the procedure, all | | |
| | disease was less than 1 cm, but did involve | | |
| | innumerable miliary disease on the subdiaphragms, | | |
| | as well as throughout all peritoneal surfaces. | | |
| HGSC* | Bilateral adnexal masses the right was approximately | Optimal primary | |
| (966) | 10 to 12 cm, left approximately 6 cm. There were | debulking to R0 | Moderate |
| | multiple small nodules within the omentum that | | |
| | appeared to be tumor. There was an approximately 2 | | |
| | cm nodule on the cecum that was sitting outside of the | | |
| | pelvis. There was a centimeter nodule sitting on the | | |
| | lower rectum in the pelvis, multiple subcentimeter | | |
| | nodules located in the lower abdomen, and the | | |
| | diaphragm on the right side was thinly coated with | | |
| | tumor that was ablated and bluntly dissected off with | | |
| | the Argon beam coagulation, so that this patient was | | |
| | optimally visually debulked to R0 disease. Pelvic and | | |
| | periaortic lymph node sampling was performed, for | | |
| | what appeared to be bulky disease in the periaortic | | |
| | lymph node bed. | | |
| HGSC | Omental mass 20 × 10 × 5 cm thick removed | Optimal | moderate |
| (995) | completely with omentectomy, bilateral adnexal | debulking to <0.5 cm | |
| | masses approximately 6 × 7 cm bilaterally, removed | | |
| | completely. Uterus slightly enlarged with an | | |
| | estimated weight of approximately 250 grams, | | |
| | removed completely, posterior carpets of peritoneal | | |
| | disease removed completely, with posterior cul-de-sac | | |
| | peritoneal stripping with argon beam to remove | | |
| | residual tumor in the pelvis down to less than 5 mm of | | |
| | residual disease. Bilateral retroperitoneal fibrosis | | |
| | necessitating dissection of the perirectal and | | |
| | perivesical spaces, and bilateral ureterolysis to allow | | |
| | complete resection of tumor and uterus secondary to | | |
| | the extensive dissection and the posterior cul-de-sac | | |
| | stripping. Diffuse 1 to 2 cm peritoneal disease | | |
| | debulked with excision or argon beam to less than 5 | | |
| | mm of residual, 5 mm thick, approximately 1 cm wide, | | |
| | scarred area at the dome of the liver in close | | |
| | approximation to the inferior vena cava which, was left | | |
| | in situ. | | |
| HGSC | Bilateral adnexal masses 4 × 5 cm on the right and | Optimal | Bright |
| (952) | 8 × 10 cm on the left. Significant retroperitoneal tumor | debulking to | |
| | burden, left greater than right necessitating | <1 cm residual | |
| | aggressive dissection and ligation of internal iliac | disease | |
| | artery to remove tumor. | | |
| HGSC | Extensive omental caking and adhered pelvis likely | Optimal | Faint- |
| (1003) | due to tumor with obliteration of the posterior cul-de- | debulking to <0.5 cm | moderate |
| | sac and the anterior cul-de-sac. Minimal normal tissue | | |
| | was able to be identified. Diaphragmatic studding and | | |
| | extensive miliary tumor disease along the mesentery | | |
| | of the small and large bowel. Otherwise, no obvious | | |
| | large tumor nodules were noted, and there was no | | |
| | lymphadenopathy. | | |
| Other | Bilateral adnexal masses as described on CT scan, | Optimal primary | Faint |
| histotype | with omental masses adherent to the uterus and the | debulking to R0 | |
| (953) | adnexal masses completely resected. The omentum | | |
| | had multiple areas of confluent tumor 3 to 4 × 4 cm in | | |
| | diameter, as well as multiple other peritoneal based | | |
| | lesions measuring anywhere between 1 to 4 cm and | | |
| | numbering approximately 100 or so. These were all | | |
| | completely removed and/or debulked with the use of | | |
| | the argon beam coagulator. There was also additional | | |
| | upper abdominal disease and splenic lesions | | |
| | completely resected by the team by consulting | | |
| | service. Debulking of the diaphragmatic lesion | | |
| | involved resection of full-thickness diaphragm, and the | | |
| | chest tube was inserted intraoperatively. | | |
| Other | Enlarged, approximately 8 to 10 cm, multicystic and | Delayed optimal | Negative |
| histotype | solid complex right adnexal mass that was densely | primary debulking | |
| (950) | adherent to the bladder, as well as an enlarged | to R0 | |
| | complex adnexal mass of the left ovary that was | | |
| | densely adherent to the left pelvic sidewall. Pelvic and | | |
| | periaortic lymph node beds were palpated, with no | | |
| | adenopathy demonstrated. Omentum had minimal | | |
| | tissue and fat, with no visible disease. The liver edges | | |
| | felt smooth, without evidence of disease bilaterally. | | |
| | There was an approximately 4 cm to 5 cm right | | |
| | diaphragmatic lesion that was removed by consulting | | |
| | surgeon. There was no other palpable or visible | | |
| | disease throughout the abdomen or pelvis. | | |
| Other | Omental cake, approximately 7 × 8 cm × | Optimal | Negative |
| histotype | approximately 4 cm thick, removed completely. Right | debulking to 1-2 mm | |
| (991) | adnexal mass, approximately 4 × 5 cm, with | | |
| | adhesions of the terminal ileum and cecum to the area | | |
| | of the mass causing upstream bowel obstruction, | | |
| | removed completely and debulked with Argon beam | | |
| | coagulator to less than 1 to 2 mm residual. Plaque of | | |
| | tumor in the deep pelvis reduced to approximately 1 to | | |
| | 2 mm plaque residual with the use of Argon beam | | |
| | coagulator. No clearly identifiable left ovary and tube; | | |
| | hence, the region of the left ovary in between the IP | | |
| | ligament above the ureter and to the pelvic sidewall | | |
| | was removed and sent together as left ovary and | | |
| | tube. | | |
| Other | Darksih brown ascites drained, approximately 9 liters. | Optimal tumor debulking | Faint |
| histotype | Enlarged bilateral adnexal masses. Uterus was small; | | |
| (993) | however, there was prior disease to the anterior cul- | | |
| | de-sac, and the bladder posteriorly in the cul-de-sac. | | |
| | There was evidence of some inflammation, as well as | | |
| | possible previous disease that was treated by | | |
| | chemotherapy. There was then an approximate 2 cm | | |
| | nodule on the omentum that was taken out with the | | |
| | omentectomy. There were also adhesions and | | |
| | thickening on the anterior abdominal wall. Liver felt | | |
| | completely smooth. Peritoneum inflamed. Small | | |
| | bowel and large bowel grossly normal. Appendix | | |
| | grossly normal. | | |
| HGSC* | Bilateral adnexal masses the right was approximately | Optimal primary | Moderate |
| (966) | 10 to 12 cm, left approximately 6 cm. There were | debulking to R0 | |
| | multiple small nodules within the omentum that | | |
| | appeared to be tumor. There was an approximately 2 | | |
| | cm nodule on the cecum that was sitting outside of the | | |
| | pelvis. There was a centimeter nodule sitting on the | | |
| | lower rectum in the pelvis, multiple subcentimeter | | |
| | nodules located in the lower abdomen, and the | | |
| | diaphragm on the right side was thinly coated with | | |
| | tumor that was ablated and bluntly dissected off with | | |
| | the Argon beam coagulation, so that this patient was | | |
| | optimally visually debulked to R0 disease. Pelvic and | | |
| | periaortic lymph node sampling was performed, for | | |
| | what appeared to be bulky disease in the periaortic | | |
| | lymph node bed. | | |

CBX2 is a subunit of the polycomb repressor complex (PRC1), which has been shown to play a role in ovarian cancer. The enzymatic subunit or “writer” of PRC1, BMI-1, is considered to play a role in malignant transformation of multiple cancers, including ovarian cancer. In ovarian cancer, BMI-1 has been demonstrated to be associated with stem-ness and tumor initiation and serves as an independent predictor of poor outcome. Moreover, silencing of BMI-1 can lead to improved sensitivity to chemotherapy. This understanding of BMI-1 directly correlates and aligns with our CBX2 findings. Taken together with observations in other types of cancer, it seems likely that the PRC1 and specifically, CBX2, are novel therapeutic targets not only for HGSOC, but potentially for breast and prostate cancers.

CBX2 is considered to be an epigenetic “reader”. The existing literature suggests that targeting epigenetic “readers” is an effective strategy for targeted therapy. Bromodomain (acetyl-histone “reader”) inhibitors have the potential to suppress ALDH activity in ovarian cancer, providing evidence that targeting of an epigenetic reader may be able to alter the stem-like phenotype of a cell. One key example is JQ-1, a potent and selective inhibitor of the bromodomain and extra-terminal domain (BET) family of proteins, including BRD2, BRD3, and BRD4. Preliminary clinical data suggests that BET inhibitors may have therapeutic potential in human cancers. Furthermore, a Phase I clinical trial of an oral BET inhibitor demonstrated that this small molecule inhibitor was well tolerated in vivo, a critical development which paves the way for future targeting of epigenetic readers. This highlights that a “reader” or chromobox domain inhibitor could prove to be more effective with fewer adverse effects compared to inhibitors of epigenetic “writer” enzymes.

Disclosed herein, Applicants describe the role of CBX2 in promoting HGSOC disease progression. Mechanistically, CBX2 protects HGSOC against apoptosis and promotes a more stem-like phenotype. CBX2 is an epigenetic reader and is therefore targetable with a small molecule inhibitor. This work expands our understanding of the progression of HGSOC and identifies a novel therapeutic target.

Increased CBX2 protein in HGSOC (compared to fallopian tube epithelium, and other histotypes) is associated with poorer prognosis for the patient, repression of the FOXO3 tumor suppressor, and chemoresistance of HGSOC tumors and cells. CBX2 expression may be determined by various methods. In some embodiments, CBX2 protein expression may be assessed by densitometry of western immunoblot study. In many embodiments, increased expression may refer to CBX2 intensity that is greater than about 10%, 20%, 30%, 40%, 50%, 60%, 70%, 80%, 90%, 100%, 110%, 120%, 130%, 140%, 150%, 200%, 300%, 400%, 500%, 600%, 700%, 800%, or 900%, and less than about 1000%, 900%, 800%, 700%, 600%, 500%, 400%, 300%, 200%, 160%, 150%, 140%, 130%, 120%, 110%, 100%, 90%, 80%, 70%, 60%, 50%, 40%, 30%, or 20% compared to CBX2 in non-HGSOC cells. In other embodiments, relative CBX2 protein expression may be determined by other methods for quantitation of protein expression that are well-known to those of skill in the art. In many embodiments, CBX2 protein may be identified by an antibody specific to CBX2, for example anti-CBX2 antibody from Thermo Fisher Scientific (Cat # PA5-30996). Relative quantitation of CBX2 may be adjusted or standardized by comparing CBX2 amounts to various control proteins, for example actin. CBX2 expression may be quantified by various methods. In some embodiments, CBX2 protein is analyzed and/or quantified by flow cytometry or mass spectrometry.

Reducing CBX2 expression, for example by various knockdown or repression methods, significantly decreases viability of HGSOC cells. In many embodiments, repression or knockdown leads to decreased growth rate and/or anoikis (anchorage-independent cell death) in HGSOC cells.

Suppression of CBX2 expression can promote cell death in HGSOC cells and/or reduce their growth rate. Suppression may be accomplished by various methods. In some embodiments, CBX2 mRNA transcripts may be targeted by one or more compounds including, but not limited to, siRNA, ribozyme, antisense, aptamer, or other small molecule. In some embodiments, CBX2 repression or knockdown may be accomplished by targeting CBX2 protein with one or more of, but not limited to, an antibody, peptide, peptidomimetic, small molecule, or other compound. In one embodiment, CBX2 transcripts may be targeted by one or more short hairpin RNA (shRNA) molecules, or other types of RNAi well-known to those of skill in the art. In some embodiments, the shRNA molecules may comprise a sequence selected from GCCAAGGAAGCTCACTGCCAT (shCBX2 #1; SEQ ID NO:19) or ACGGAAAGGAACAGGAAGCAT (shCBX2 #2; SEQ ID NO:20). In many embodiments, HGSOC cells may be induced to reduce their growth rate or enter anoikis by reducing the amount of CBX2 transcripts by greater than about 5%, 10%, 20%, 30%, 40%, 50%, 60%, 70%, 80%, 90%, or 95%, and less than about 100%, 90%, 80%, 70%, 60%, 50%, 40%, 30%, 20%, or 10% compared to HGSOC cells that have not been treated to reduce their CBX2 expression.

Patients having HGSOC cells with enhanced expression of CBX2 indicates that chemotherapy may be less effective. In some embodiments, the chemotherapy is a platinum-based compound, for example selected from one or more of cisplatin, carboplatin, and oxaplatin. In some embodiments, patients whose HGSOC tumor cells express high levels of CBX2 require an increased dosage of chemotherapeutic drug of greater than 10%, 20%, 30%, 40%, 50%, 60%, 70%, 80%, 90%, 100%, 150%, 200%, 250%, 300%, or 400%, and less than about 500%, 400%, 300%, 200%, 1500%, 100%, 90%, 80%, 70%, 60%, 50%, 40%, 30%, or 20%, compared to patients with tumors having lower CBX2 expression.

Repression of CBX2 expression can increase sensitivity of HGSOC cells to chemotherapy. In some embodiments, various therapies may be used to reduce expression of CBX2 in HGSOC cells and tumors. This may allow the use of various chemotherapeutic compounds to treat a patient positive for HGSOC and/or may allow the use of lower doses of chemotherapy to achieve a beneficial therapeutic effect, such as a reduction in tumor growth, lower cell growth, an increase in cancer cell death, or reduction in adverse side effects such as fatigue, hair loss, bruising, bleeding; infection; low red blood cell counts; nausea, etc.

CBX2 expression correlates to mRNA expression of genes associated with apoptosis, autophagy, and epithelial to mesenchymal transition (EMT). In many embodiments, the genes may include one or more of MYLK, NOG, and TNFSF10

Increased CBX expression results in driving cells toward a stem-like phenotype. In many embodiments, a stem-like phenotype may be evaluated by measuring aldehyde dehydrogenase (ALDH), where stemness is associated with increased ALDH activity. In many embodiments, CBX2 expression correlates with ALDH3A1 expression, and knockdown of CBX2 expression decreases ALDH3A1 expression, as well as the stem cell-associated transcription factor, SOX4.

### Definitions

Biomarker may refer to any measurable indicator of the state of a cell, tissue, organ, or subject. In some embodiments, the biomarker is a molecule associated with a cell, for example a protein, peptide, or mRNA transcript. In some embodiments, the protein may be full-length or truncated protein, or a peptide fragment of a full-length protein. In some embodiments, a biomarker may be a nucleic acid, such as an mRNA transcript. In many embodiments, the biomarker may be an mRNA or protein associated with a given gene, for example CBX2, SOX4 MYLK, NOG, and TNFSF10. In most embodiments, the biomarker is a mammalian biomarker, for example human biomarker.

Subject includes any mammal, including mice, rats, guinea pigs, rabbits, dogs, cats, cows, horses, monkeys, and humans. A patient is a subject undergoing treatment or observation for a condition or disease, such as cancer. Cancer includes conditions or diseases of mammals characterized by uncontrolled cellular growth, hyperproliferative growth, hyperplasic growth, neoplastic growth, cancerous growth or oncogenic processes. Cancer may also refer to tumors, metastatic tissues or malignantly transformed cells, tissues, or organs, irrespective of histopathologic type or stage of invasiveness.

Chemotherapeutic agents include synthetic, natural, semisynthetic compounds and molecules useful in treating, killing, suppressing, or controlling cells that are displaying uncontrolled growth. Chemotherapeutic agents may reduce the proliferation of such cells and/or induce their death (programmed or non-programmed cell death). Chemotherapy refers to administration of such agents to a patient in need thereof. Chemoresistance may refer to the ability of a cell to avoid death or a decrease in proliferation in the presence of a chemotherapeutic agent.

Biological sample may refer to any sample removed, extracted, or derived from a patient or subject. In many embodiments a biological sample includes tissues, cells, protein, nucleic acids, etc. A biological sample may be processed for viewing or analysis, such as protein or nucleic acid quantitation. A control sample or reference sample may be a sample obtained from a similar subject, tissue, or cell that does not have, or is not expected to have, a disease or condition that is being assayed. In some embodiments, a reference sample may include samples from two or more subjects.

Therapeutically effective, as used herein refers to a therapeutic treatment, for example administration of a chemotherapeutic agent, that is adequate to accomplish a desired, expected, or intended result. When used in the context of treating a patient or subject, it may refer to an amount of agent which, when administered to a subject or patient for treating or preventing a disease, is an amount sufficient to effect such treatment or prevention of the disease or condition, and/or ameliorates at least one consequence of the disease or condition.

Inhibition or repression, as used herein means to lessen by some amount. For example, inhibition of gene expression by a compound or agent may cause the amount of protein or transcript in a given cell to be reduced relative to a cell that has not been contacted with the compound or agent.

Antibody may refer to any natural, synthetic, human, non-human, or humanized protein that may bind to an epitope on a target antigen. Antibodies may be mono or bi-specific. Antibodies may be multi or single chain proteins. In some embodiments the antibodies may include one or more Fc domains, Fv domains and/or CDRs. Antibodies may refer to peptides or peptide mimetics that bind to a target protein.

RNA interference (RNAi) includes any method of targeting expression of one or more genes by degrading mRNA transcribed from the gene. RNAi include siRNA, miRNA, shRNA, ribozyme, aptamers and the like, which typically contain some amount of sequence complementary to the gene transcript. RNAi techniques and methods are well known to those of skill in the art.

Peptide, polypeptide, and protein fragment may refer to natural or synthesized compounds and molecules containing natural or synthetic amino acids, amino acid equivalents and/or other non-amino groups. Peptides may be modified by replacement of one or more amino acids with related compounds, and/or modified by removing or adding elements, compounds, or molecules to one or more side chains or functional groups. The peptides can be linear or cyclic. Peptidemimetic refers to a compound or molecule that mimics a peptide in the peptide's ability to assume a three-dimensional structure on its own, or as a result of binding or contacting an epitope.

Metastasize may refer to the ability of a cancer cell to travel from the primary tumor, proliferate, and establish a second tumor. In some embodiments, a metastatic cell may be able to proliferate in suspension without attaching or adhering to a substrate, for example connective or other solid non-cancerous tissue.

### Examples

### Example 1—CBX2 is Upregulated in High Grade Serous Ovarian Cancer and is Associated with Poor Survival

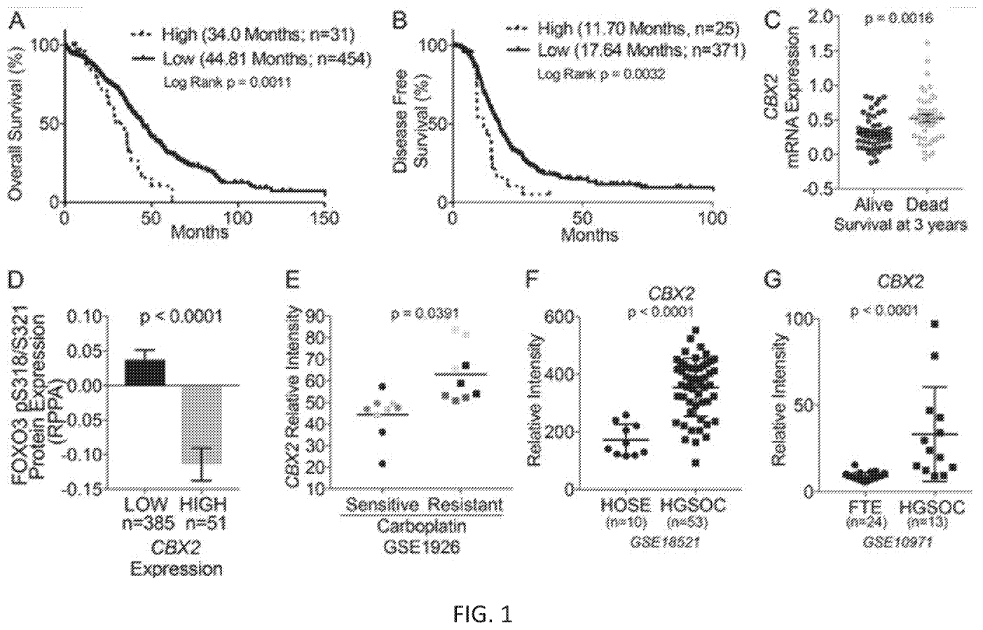

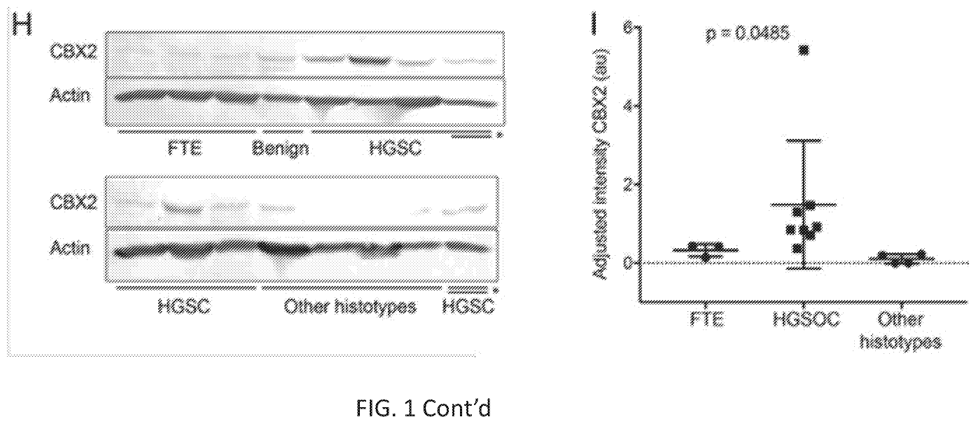

We examined CBX2 expression in HGSOC in several publicly available datasets (Gene Expression Omnibus; GEO Dataset and The Cancer Genome Atlas; TCGA). High expression of CBX2 in TCGA HGSOC samples conveyed both a significantly worse disease-free survival (DFS; 11.7 vs. 17.6 months, Log-rank test p-value 0.00316) and overall survival (OS; 34 vs. 44.8 months, Log-rank test p-value 0.00116) (FIG. 1 Panels A and B). In an independent HGSOC data set, high CBX2 expression was associated with poorer survival at 3 years (FIG. 1 Panel C). Further correlation of CBX2 expression with protein expression via reverse-phase protein array (RPPA) found several proteins significantly enriched or depleted in CBX2 high expressing tumors (Table 1). Notably, phosphorylated serine 318 and 321 FOXO3, a known tumor suppressor, was depleted in tumors with high CBX2 expression (FIG. 1 Panel D). Additionally, using the GEO Dataset (GSE1926), a comparison of platinum sensitive HGSOC tumors to platinum resistant HGSOC tumors, demonstrated an increase in CBX2 in resistant tumors, further supporting the association between CBX2 and more aggressive HGSOC (FIG. 1 Panel E).

FIG. 1. Panels are as follows. Panel A Overall survival analysis comparing expression of CBX2 (High=mRNA expression >1.5 standard deviation; Low=SD<1.5(=)). High (n=31) vs. Low (n=454) CBX2 expression (mean survival 34.0 months vs. 44.81 months, Log Rank p=0.0011). Panel B Same as (Panel A), disease-free survival analysis, again comparing upregulation of CBX2, defined as above (High n=25, Low n=371). Increased expression of CBX2 was associated with statistically significant decreased disease-free survival (11.70 months vs. 17.64 months, Log Rank p=0.0032). Number of patients differ from (Panel A) due to available data within TOGA. Panel C Examination of CBX2 mRNA expression in HGSOC patients alive (n=51) and dead (n=42) at 3 years after diagnosis. Data obtained from HGSOC Tothill Cohort. Statistical test=two-sided t-test, F test p=0.0057. Panel D Correlation of CBX2 expression with FOXO3_PS318/321 in TOGA (“Provisional data” with RPPA data, n=435) tumors (High=upper quartile and Low=bottom three quartiles). Statistical test=two-sided t-test, F test p<0.0001. Panel E Bioinformatic analysis evaluating relative intensity of CBX2 in high grade serous ovarian carcinoma (HGSOC) cases described as platinum sensitive (n=3) or resistant (n=3). Each tumor (color-coded) was examined in triplicate. Statistical test examined average CBX2 intensity for each tumor (GSE1926; one-sided t-test p=0.0391, F test p=0.29). Panel F Relative intensity of CBX2 in benign human ovarian surface epithelium (HOSE, n=10) compared to HGSOC, n=53 (GSE18521; t-test p<0.0001, F test p<0.0001). Panel G Relative intensity of CBX2 in fallopian tube epithelium (FTE, n=24) compared to HGSOC, n=13 (GSE10971; two-sided t-test p<0.0001, F test p<0.0001). Panel H Protein lysates generated from the primary tissue of FTE, HGSOC, and mixed histotypes. Protein utilized for immunoblot against CBX2. (beta-actin=loading control). Panel I Densitometric analysis of immunoblots. Intensities were normalized between immunoblot by indicated (*) sample (FTE vs. HGSOC, one-sided Rank Sum p=0.0333)

Ovarian surface and FTE are proposed to be the precursor cells for HGSOC; more recent data strongly support FTE as the predominant site of origin. Comparing CBX2 expression in ovarian surface epithelium or FTE to CBX2 expression in HGSOC, we observed that CBX2 was significantly higher in HGSOC (FIG. 1 Panels F and G) (GSE18521 and GSE10971). To confirm the extent of these findings we examined protein derived from primary tissues of four FTE and benign tissues and seven HGSOC tumors collected through the University of Colorado Gynecologic Tumor and Fluid Bank (GTFB) (FIG. 1 Panel H; Table 2). Utilizing densitometry, CBX2 expression was observed to be significantly higher in HGSOC primary tumor compared to FTE or benign tissues (FIG. 1 Panel I, Rank-sum test p value 0.0333). In other ovarian cancer histosubtypes, we noted a lack of CBX2 expression suggesting the oncogenic effects of CBX2 are HGSOC specific. Taken together, these data demonstrate that CBX2 upregulation in HGSOC is associated with poorer prognosis, repression of the FOXO3 tumor suppressor, and is possibly linked to chemoresistance.

### Example 2—CBX2 is Upregulated in Tumor Cells in Suspension

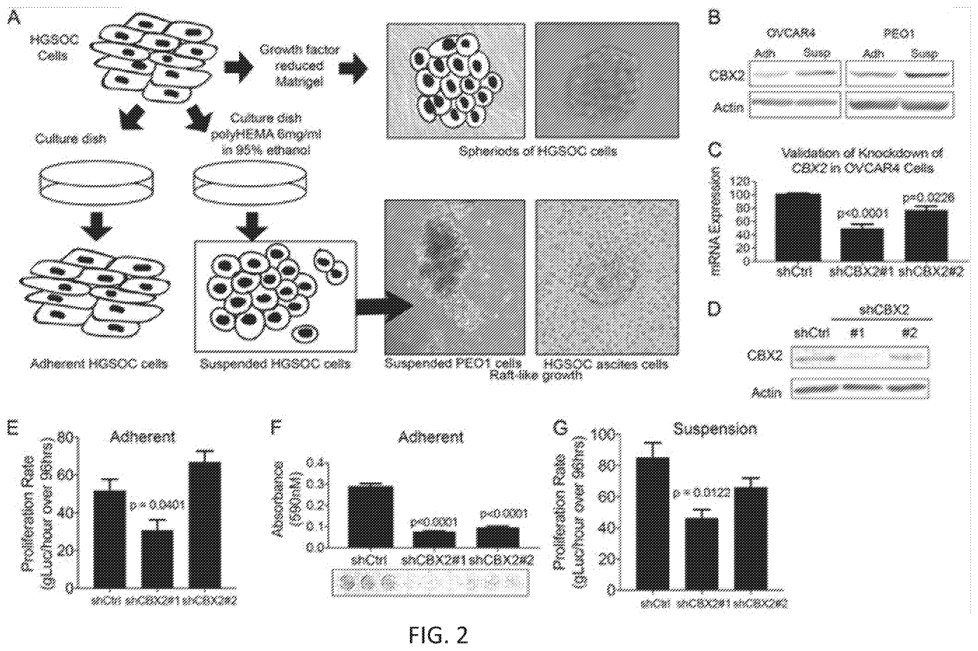

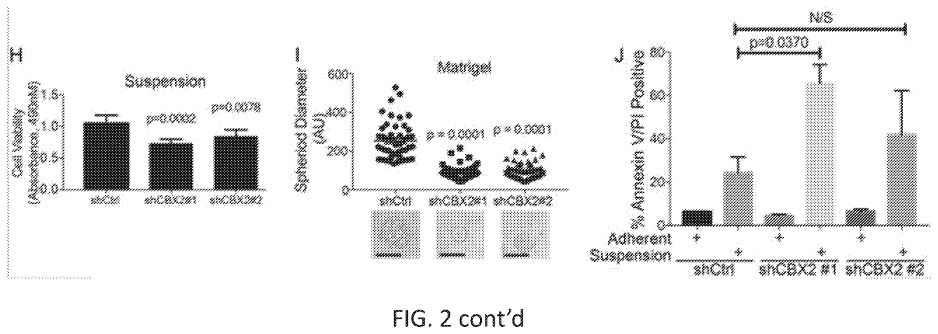

HGSOC is unique compared to other solid types in its tendency to directly seed and disseminate throughout the peritoneal cavity, which requires an escape from anoikis, an anchorage-independent cell death. To determine whether CBX2 plays a role in HGSOC tumor cell's ability to survive without anchorage, or in a suspended setting, we examined the role of CBX2 on HGSOC growth in suspension. A forced suspension setting was achieved by plating cells on polyHEMA-coated tissue culture dishes (FIG. 2 Panel A).

FIG. 2 Panels are as follows. Panel A Model describing basic protocol for establishing adherent, suspension, and spheroid growth environments. For adherent and suspension, two verified high grade serous ovarian carcinoma cell lines were initially grown on tissue culture plastic, then distributed to normal tissue culture dishes (adherent) and polyHEMA coated culture dish (suspension) growth environments. Distributed at 1:3 or 1:5 ratio to account for forced suspension induced cell death. Photographs show PEO1 cells after 7 days in suspension (left) and HGSOC cells directly derived from patient ascites. For spheroid formation, cells were grown in 3D in Matrigel for 12 days. A representative image of a resulting spheroid is shown at upper right. Panel B Immunoblots against CBX2 protein from OVCAR4, and PEO1 cells grown in adherent and suspension settings (described in (Panel A)) over 7 days. Panel C RT-qPCR for CBX2 in OVCAR4 cells transduced with small hairpin RNA (shRNA) specific for CBX2. Representative mRNA expression of CBX2 in shControl, shCBX2#1, and shCBX2#2. Statistical test=ANOVA. Panel D Immunoblots against CBX2 protein derived from OVCAR4 shControl, shCBX2#1, and shCBX2#2 transduced cells (beta-actin=loading control). Panel E Proliferation assay of OVCAR4 cells with CBX2 knockdown and shControl, grown in adherent setting (tissue culture plastic) over 96 h, evaluated using gLuc activity. Statistical test=ANOVA. Panel F Same as (e), crystal violet staining and subsequent measurement of absorbance at 590 nm. Images of representative of stained cells from shControl, shCBX2#1, and shCBX2#2. Statistical test=ANOVA. Panel G Proliferation assay of OVCAR4 cells with CBX2 knockdown and shControl, grown in suspension setting (poly-HEMA coated tissue culture plastic) over 96 h, evaluated using gLuc activity. Statistical test=ANOVA. Panel H Same as (g), but cell viability was assessed via MTT after 96 h. Statistical test=ANOVA. Panel I OVCAR4 cell lines (shControl, shCBX2 #1 and #2) grown in 3D using Matrigel over 12 days, leading to spheroid growth. Representative images of transduced OVCAR4 cells below. Scale bar=100 μm. Spheroids measured across horizontal diameter. Diameter mean calculated from measurements of 50 spheroids per cell type. Statistical test=ANOVA. Panel J OVCAR4 cells with CBX2 knockdown and shControl grown in adherent or suspension. Cells were subjected to AnnexinV/PI apoptosis assay. Statistical test=two-sided t-test, F test p=0.9291. Error bars=S.E.M.

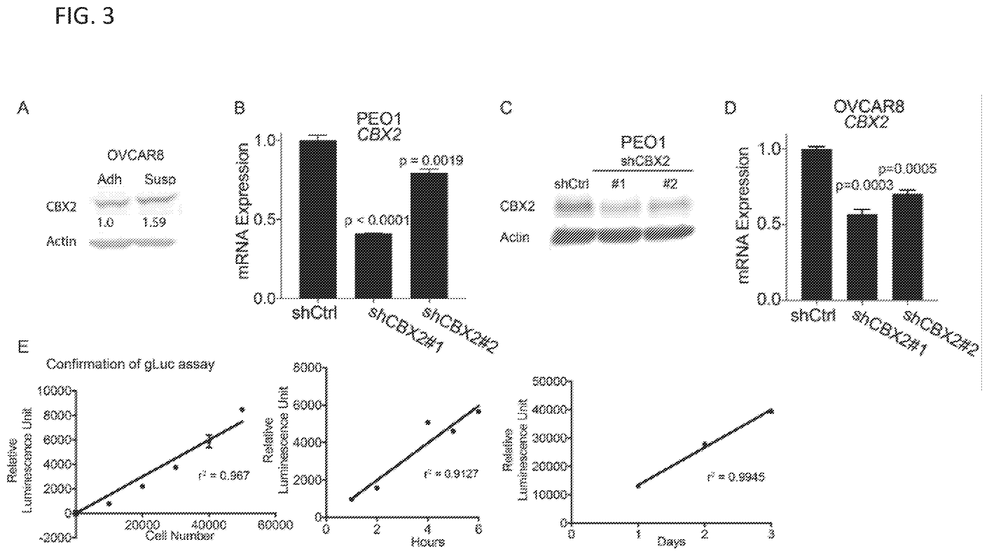

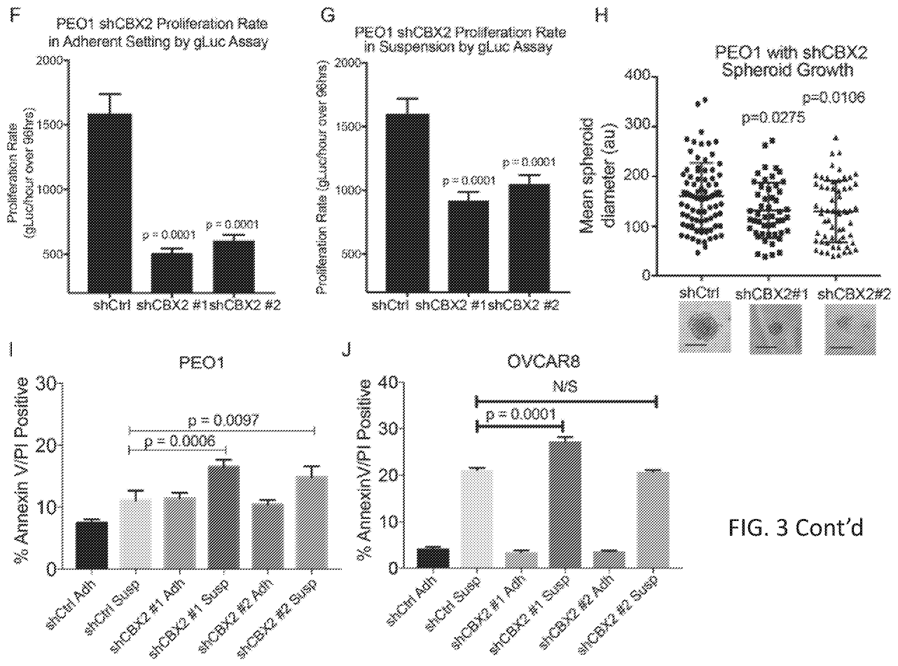

The high grade serous ovarian cancer cell lines OVCAR4, PEO1, and OVCAR8 were grown in adherent and suspended settings (FIG. 2 Panel A). During the course of optimizing our model, we noted that OVCAR4 and PEO1 cells grown in suspension demonstrated a morphology and organization similar to cells derived from primary ascites fluid (data not shown and FIG. 2 Panel A). After culturing HGSOC cell lines for 7 days in a forced suspension, condition protein was extracted and subsequently used for immunoblots against CBX2. For all cell lines examined, CBX2 expression was increased in cells grown in suspension conditions (FIG. 2 Panel B; and FIG. 3 Panel A). This observation serves as the foundation for our work, as the phenotype we describe is reinforced by both cell survival in suspension, as well as intact expression of CBX2.

The panels of FIG. 3 are as follows. Panel A: OVCAR8 grown in adherent and suspension settings (described in FIG. 2A) over 7 days. Protein utilized for immunoblot against CBX2. Panel B: RT-qPCR for CBX2 in PEO1 cells transduced with small hairpin RNA control (shCtrl) or specific for CBX (shCBX2 #1 and #2). Internal control=18s. Statistical test=ANOVA. Panel C: Protein derived from PEO1 shControl (shCtrl), shCBX2#1, and shCBX2#2 transduced cells utilized for immunoblot against CBX2 (actin=loading control). Panel D: Same as B, but examined CBX2 mRNA expression in OVCAR8 cells. Internal control=18s. Statistical test=ANOVA. Panel E: Experimental confirmation of Gaussia luciferase (gLuc) assay. OVSAHO cells were transduced with gLuc virus and selected with puromycin. Known number of cells grown over 24 hours, media collected, gLuc assay performed (left). Equivalent number of cells seeded across 24 well plate, media collected and assayed over hours (middle) and days (right). Relative luminescence units by gLuc increases with number of cells. Linear regression r² indicated. Panel F: Proliferation assay of PEO1 cells with CBX2 knockdown and scramble control, grown in adherent setting (tissue culture plastic), measured by gLuc activity every 24 hr for 96 hr. Calculated as proliferation rate: gLuc relative intensity per hour. Statistical test=ANOVA. Panel G: Same as E, PEO1 cells grow in suspension setting with utilization of polyHEMA coated plates (FIG. 2A). Proliferation rate calculated. Statistical test=ANOVA. Panel H: PEO1 cell lines (shControl [shCtrl], shCBX2 #1 and #2) grown in 3D using Matrigel over 12 days, forcing spheroid growth. Spheroids measured across horizontal diameter. Mean calculated from all measurements. Representative images of spheroids below X axis. Scale Bar=100 μm. Statistical test=ANOVA. Panel I: ShCtrl, shCBX2 #1 and #2 PEO1 cells grown in adherent or suspension were utilized for an AnnexinV/PI assay. Percentage positive AnnexinV/PI graphed. Statistical test=ANOVA. Panel J: Same as I, but examined OVCAR8 cells. Error bars=S.E.M.

### Example 3—CBX2 Knockdown Inhibits Proliferation

To further elucidate the role of CBX2 in HGSOC, we evaluated the impact of CBX2 modulation in PEO1, OVCAR4, and OVCAR8 HGSOC cell lines. One of the two independent small hairpin RNAs (shRNA) specific for CBX2 or a control (shControl) were transduced into OVCAR4, PEO1, and OVCAR8 cells. CBX2 knockdown was confirmed via quantitative PCR (qPCR) and immunoblots, with approximately 60% knockdown in the presence of shCBX2#1 and 30% knockdown in the presence of shCBX2#2 (FIG. 2 Panels C, D; and FIG. 3 Panels B-D). PEO1 and OVCAR4 CBX2 knockdown cells were subjected to proliferation assays in 2D tissue culture dishes and in suspension as demonstrated in FIG. 2 Panel A. To assess changes in proliferation, cells were transduced with a retrovirus specific for Gaussia luciferase (gLuc). Changes in gLuc activity were shown to be directly correlated with cell number (FIG. 3 Panel E). OVCAR4 and PEO1 CBX2 knockdown cells were plated in adherent (2D) conditions and for 96 h gLuc activity was measured every 24 h. As a confirmatory assay, colony formation was examined in parallel on cells grown in 2D. CBX2 knockdown cells had a significantly reduced rate of gLuc activity and reduced colony formation (FIG. 2 Panels E and F and FIG. 3 Panel F). OVCAR4 and PEO1 CBX2 knockdown cells were plated in forced suspension conditions and gLuc activity was monitored every 24 h for 96 h and cell viability was determined for cells grown in forced suspension. Similar to adherent conditions, CBX2 knockdown had a significantly reduced rate of gLuc activity and viability (FIG. 2 Panels G and H and FIG. 3 Panel G). HGSOC grown on extracellular matrix more closely recapitulates the tumor microenvironment, therefore OVCAR4 and PEO1 shControl and shCBX2 (#1 and #2) cells were grown in matrigel for 12 days. Spheroid diameter was measured for at least 50 spheroids in each condition and used as a surrogate for cell number. CBX2 knockdown significantly reduced spheroid size compared to shControl control cells (FIG. 2 Panel I and FIG. 3 Panel H). In all culture conditions examined, we observed that CBX2 knockdown significantly decreased HGSOC cell viability. Upon closer examination of forced suspension conditions, we observed in OVCAR4, PEO1, and OVCAR8 cells that CBX2 knockdown potentiated anoikis, anchorage-independent cell death (FIG. 2 Panel J and FIG. 3. Panels I-J). These data indicate that CBX2 is important in promoting HGSOC cell proliferation and protecting against anoikis.

### Example 4—Tissue Microarray Supports a Role for CBX2 in Tumor Progression

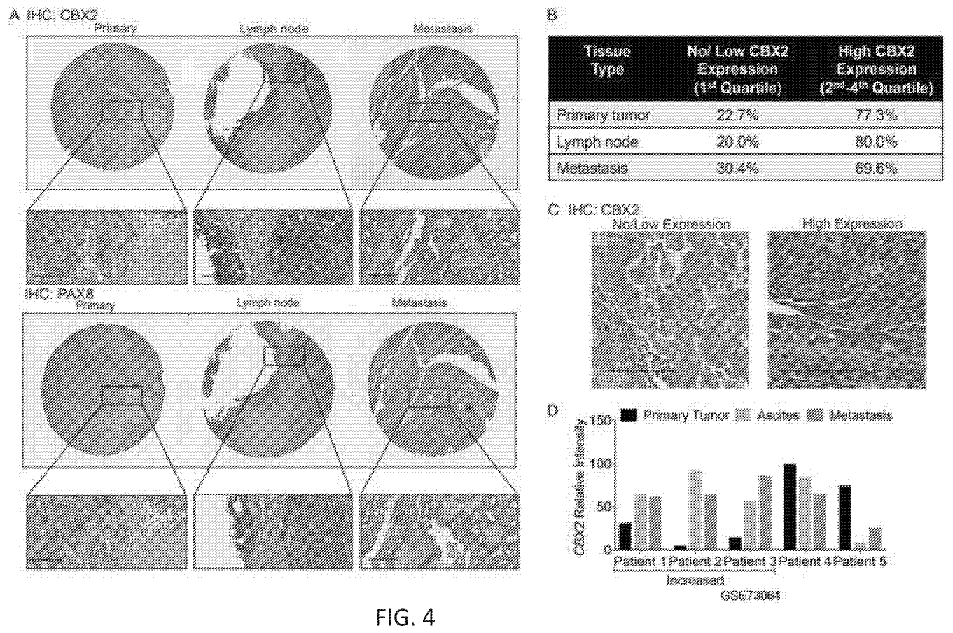

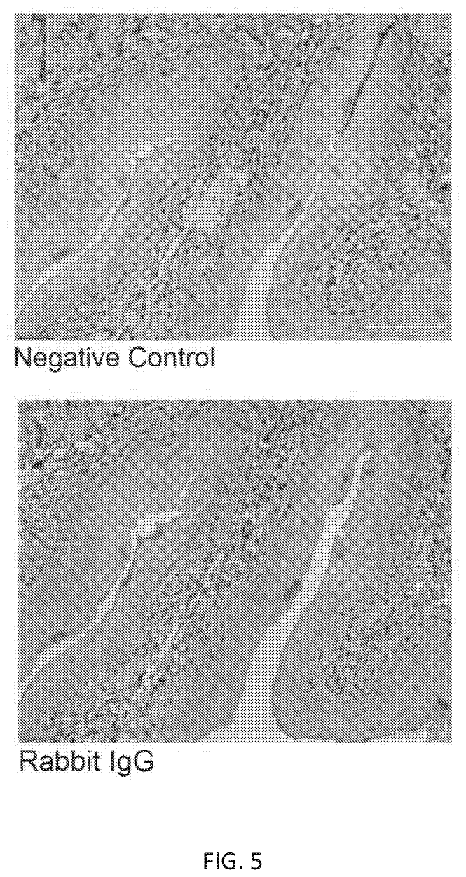

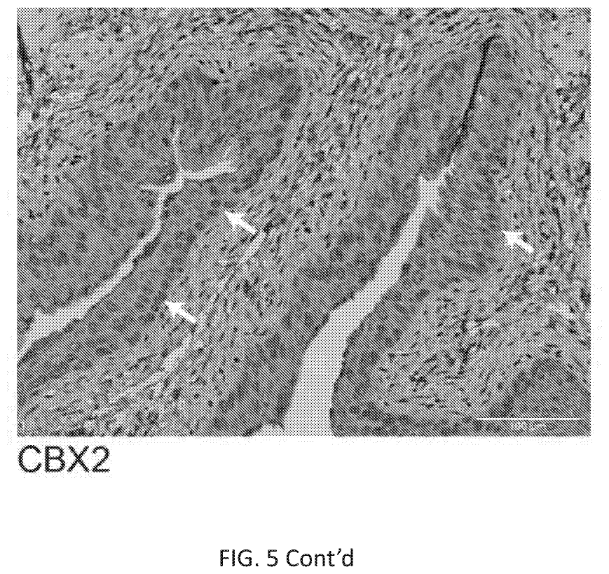

In order to correlate the CBX2 in vitro findings to clinically relevant specimens, we utilized a tissue microarray (TMA) of HGSOC that recapitulated tumor progression. The TMA contained 24 primary tumors, with matched lymph node or distant metastases (Table 3). IHC was optimized and performed using a previously published CBX2 antibody, predicted to preferentially stain the nucleus (FIG. 4 Panel A, and FIG. 5) 13.

TABLE 3

| Clinical | | | | | | |
|---|---|---|---|---|---|---|
| characteristics | | | | | | |
| of matched | | | | | | |
| tissue | | | | | % Positive | |
| microarray | | | | % Positive | Stain | % Positive |
| Age at | Stage at | | | Stain | Lymph | Stain |
| diagnosis | diagnosis | Pathologic diagnosis | PDS vs NACT | Primary | node | Metastasis |
| No clinical data | | | | 36.51% | 22.09% | 27.44% |
| available | | | | | | |
| No clinical data | | | | 20.68% | 26.93% | 33.06% |
| available | | | | | | |
| No clinical data | | | | No | No | 45.85% |
| available | | | | primary | primary | |
| 36 | IV | [Serous carcinoma, FIGO | PDS (optimal) | 34.30% | 53.14% | 10.75% |
| | | Grade III per clinical | | | | |
| | | documentation] | | | | |
| 59 | IIIC | Papillary serous | PDS (optimal) | 50.23% | 29.23% | 24.21% |
| | | carcinoma | | | | |
| 84 | IIIC | Papillary serous | PDS (optimal) | 32.84% | 23.91% | 18.66% |
| | | carcinoma | | | | |
| 46 | IIIC | Papillary serous | PDS (optimal) | 14.40% | 26.76% | 15.55% |
| | | carcinoma | | | | |
| 73 | IIIC | Serous carcinoma, FIGO | PDS (optimal) | 6.96% | n/a | 18.72% |
| | | Grade III | | | | |
| No clinical data | | | | 32.60% | 50.16% | n/a |
| available | | | | | | |
| 67 | IIIC | Poorly differentiated | PDS (optimal) | 26.75% | n/a | 13.88% |
| | | high-grade (FIGO III) | | | | |
| | | multifocal serous | | | | |
| | | cystadenocarcinoma | | | | |
| 39 | IV | Moderate to poorly | PDS (optimal) | 30.24% | 23.62% | 30.78% |
| | | differentiated serous | | | | |
| | | papillary | | | | |
| | | cystoadenocarcinoma | | | | |
| | | (FIGO III) | | | | |
| 44 | IIIC | Serous carcinoma, FIGO | PDS (optimal) | 2.77% | 3.43% | 11.49% |
| | | grade III | | | | |
| 50 | IIIC | Papillary serous | PDS (optimal) | 14.96% | n/a | 12.52% |
| | | carcinoma, high grade | | | | |
| 63 | IIIC | Poorly differentiated | PDS (optimal) | n/a | 16.82% | n/a |
| | | serous carcinoma | | | | |
| 54 | IIIC | Serous carcinoma (FIGO | PDS (optimal) | 33.47% | 9.53% | 31.03% |
| | | Grade III) | | | | |
| 74 | IIIC | Poorly differentiated | PDS (optimal) | n/a | 13.43% | 27.78% |
| | | serous carcinoma, Grade | | | | |
| | | III | | | | |
| 66 | IVA | Serous carcinoma, | PDS (optimal) | 19.07% | 34.20% | 2.76% |
| | | Grade III | | | | |
| No clinical data | | | | 19.27% | 27.81% | 17.52% |
| available | | | | | | |
| 54 | IIIC | High grade serous | PDS (optimal) | 31.67% | 28.51% | 25.01% |
| | | carcinoma | | | | |
| 50 | IIIC | Poorly differentiated | PDS (optimal) | 27.17% | 45.78% | 25.94% |
| | | serous carcinoma | | | | |
| 60 | IVB | High grade serous | PDS (suboptimal) | 23.51% | 18.58% | 24.09% |
| | | carcinoma | | | | |
| 51 | IIIC | High grade serous | PDS (optimal) | 67.66% | 38.10% | 33.35% |
| | | papillary carcinoma | | | | |
| 66 | IVB | High grade serous | PDS (optimal) | 42.95% | 50.99% | 32.12% |
| | | carcinoma | | | | |
| 70 | IVA | Poorly differentiated | PDS (suboptimal) | 15.81% | n/a | 23.21% |
| | | serous carcinoma | | | | |
| 63 | IIIC | High grade serous | PDS (suboptimal) | 36.87% | n/a | 25.52% |
| | | carcinoma | | | | |

In parallel the TMA was stained with PAX8, a marker for Müllerian origin (FIG. 4). CBX2 and PAX8 stained TMAs were scanned using the Aperio system and annotated based on the PAX8 staining profile. Objective software-based approaches were utilized to score and analyze the scanned and annotated CBX2 TMAs. The level of expression was compared between primary tumors, metastases, and lymph nodes and quartiles were calculated. Across all three tissue types, 20-30% of the specimens were considered “no or low expression” (first quartile) compared to 69-80% of the tissues that were moderate to high expression (second through fourth quartile) (FIG. 4 Panels B and C). Results from Aperio analysis were confirmed via manual scoring by two independent board-certified pathologists. Similarly, when the intensity of staining was evaluated between matched samples there was no significant change between matched samples. Given that the tissues were derived from HGSOC patients with advanced disease, as demonstrated by both metastatic and lymph node involvement, this would suggest that CBX2 expression potentially correlates with a more aggressive disease. We next examined CBX2 expression in matched primary tumor, ascites-associated tumor cells, and metastatic tumors from five HGSOC patients (GSE73064). In two of the five patients, CBX2 expression was high in the primary tumor, however three of the five patients had an increase of CBX2 in metastatic and ascites samples compared to primary tumor (FIG. 4 Panel D). Consistently, these findings align with our bioinformatics analysis indicating that increased CBX2 expression is associated with decreased survival and more advanced disease.

FIG. 4 panels are as follows. Panel A Immunohistochemistry (IHC) against CBX2 and PAX8 utilizing a HGSOC tissue microarray (TMA) of 24 matched patient samples. Representative images of matched patient samples shown. Initial images shown at 3×, inset of images at 18×. Scale bar=100 μm. Panel B TMA analyzed using PAX8 staining a control for tumor area followed by analysis with Image Scope software to determine relative CBX2 tumor-associated intensity. Level of expression divided into quartiles. Breakdown of samples into 1st quartile (low or no expression) compared to 2nd-4th quartiles (moderate or high expression). Analysis by two board-certified pathologists (authors: M.D.P. and A.A.B.) confirmed Aperio findings. Values displayed in table, CBX2 expression seems to be consistent between tissue types. Panel C Representative images of “High” and “No/Low” CBX2 expression. Scale bar=100 μm. Panel D Bioinformatic analysis evaluating relative intensity of CBX2 in high grade serous ovarian carcinoma (HGSOC) cases with matched primary tumor (black bars), ascites-associated tumor cells (Ascites, light gray bars), and distant metastasis (dark gray bars) (n=5, GSE73064).

FIG. 5 shows serial sections of a HGSOC tumors were utilized for IHC against a negative control, a matched isotype (Rb IgG), and CBX2 (brown). As expected a majority of the CBX2 localized to the nucleus (white arrows). Scale bars=100 μm.

### Example 5—CBX2 Expression is Associated with Chemoresistance

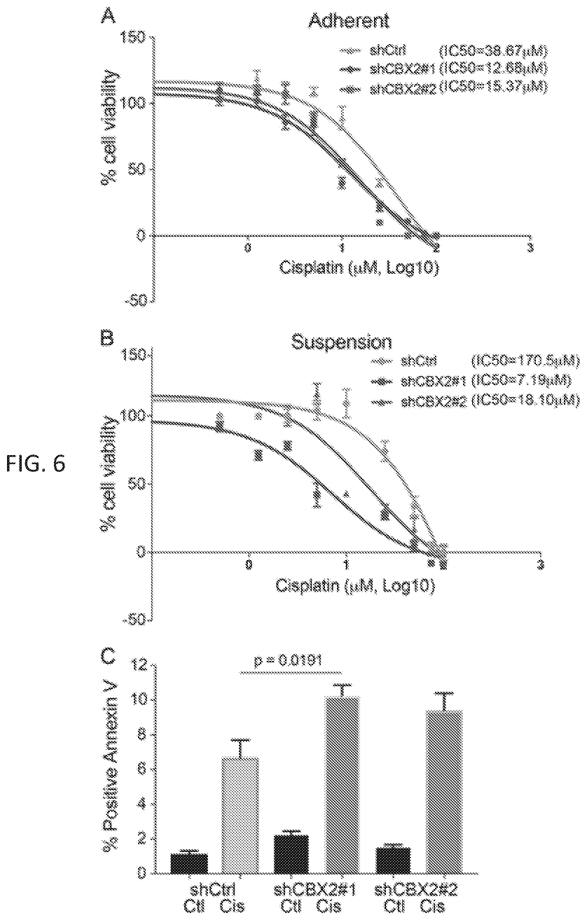

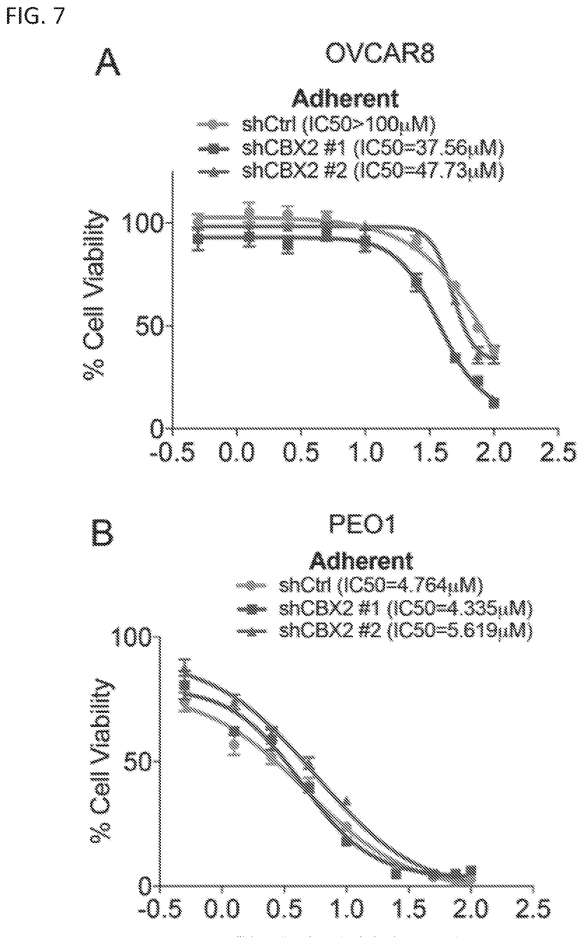

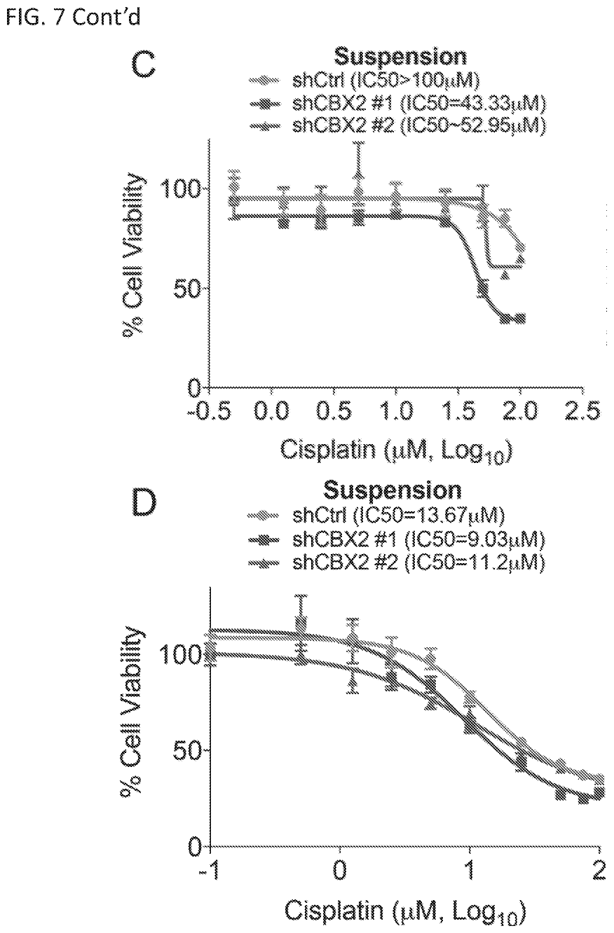

A majority of HGSOC patients treated with platinum-based (i.e., carboplatin) chemotherapy develop chemoresistance. A comparison of carboplatin sensitive HGSOC tumors to platinum resistant tumors demonstrates an increase in CBX2 in platinum resistant tumors (GSE1926) (FIG. 1 Panel C). Based on these data and our finding that CBX2 protects against anoikis, we hypothesized that CBX2 attenuates chemotherapy response. To test this hypothesis, CBX2 knockdown OVCAR4, PEO1, and OVCAR8 cells were grown in an adherent setting for 24 h and subsequently treated with increasing doses of cisplatin. Assessment of cell viability showed that in OVCAR4, PEO1, and OVCAR8, shCBX2 cell lines were significantly more chemosensitive than the shControl cells (FIG. 6 Panel A and FIG. 7 Panels A-B).

For example, CBX2 knockdown OVCAR4 cells had an 1050 of 12.68 μM in shCBX2#1 and 15.37 μM in shCBX2#2 compared to 38.67 μM for the control with intact CBX2 (FIG. 6 Panel A). Haley et al. reported the OVCAR4 cisplatin 1050 to be approximately 6 μM, however unlike this report we did not allow cells to recover 72 h following cisplatin treatment which likely accounts for this discrepancy. CBX2 knockdown OVCAR4, PEO1, and OVCAR8 cells were grown in suspension and dosed with cisplatin. OVCAR4 shCBX2 cell lines grown in suspension were found to be re-sensitized to platinum treatment with an 1050 of 7.19 μM (shCBX2#1) and 18.10 μM (shCBX2#2) compared to the control with intact CBX2 at an 1050 of 170.50 μM (FIG. 6 Panel B). Although not as robust as OVCAR4 cells, CBX2 knockdown also sensitized OVCAR8 and PEO1 cells to cisplatin (FIG. 7 Panels C-D). Notably, in OVCAR4 cells we observed a 4.47-fold increase in the cisplatin 1050 in suspension cells compared to the adherent cells. In addition, we further confirmed that in OVCAR4, loss of CBX2 lead to increased cisplatin-induced apoptosis measured with Annexin V/PI (FIG. 6 Panel C). These findings strongly support the hypothesis that anoikis-resistance, demonstrated by survival in suspension, and intact CBX2 expression, both promote chemoresistance.

Panels of FIG. 6 are as follows. Panel A OVCAR4 shControl, shCBX2#1, and shCBX2#2 in 96-well plates treated over 24 h with increasing dose of cisplatin (0.5-100 μM). Percent cell viability was measured using the MTT assay and the half maximal inhibitory concentration (1050) calculated. Panel B Similarly to (Panel A), OVCAR4 knockdown cell lines were grown in low adherent 96-well plates (forced suspension) and treated with increasing doses of cisplatin over 24 h and percent cell viability measured with MTT for calculation of 1050. Panel C Annexin/V apoptosis assay of OVCAR4 cells grown in adherent setting with shControl, shCBX2#1 or #2 treated with cisplatin (10 μM) compared to untreated control. Percent Annexin positive cells are shown. Statistical test=ANOVA. Error bars=S.E.M.

The panels in FIG. 7 are as follows. Panel A: shControl (shCtrl), shCBX2 #1 and #2 OVCAR8 cells grown in adherent were dosed with cisplatin for 24 hours. Treated cells were utilized for a MTT assay to assess cell viability. Panel B: Same as A, but adherent PEO1 cells were dosed with cisplatin for 48 hours. Panel C: Same as A, but OVCAR8 cells in suspension were dosed with cisplatin for 24 hours. Panel D: Same as A, but PEO1 cells in suspension were dosed with cisplatin for 48 hours. 1050 values were calculated with Prism and are indicated.

### Example 5—CBX2 Regulation of Autophagy, Apoptosis, and EMT-Related Genes

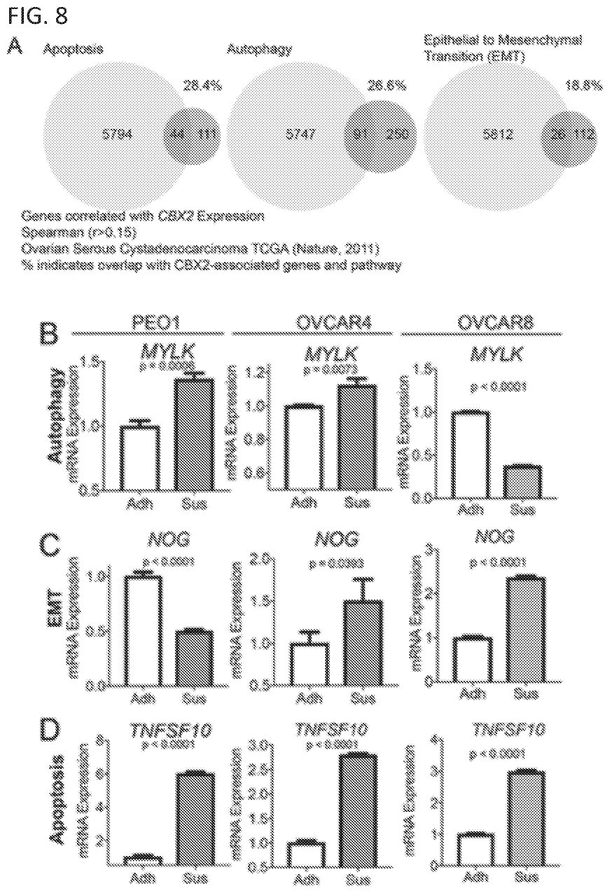

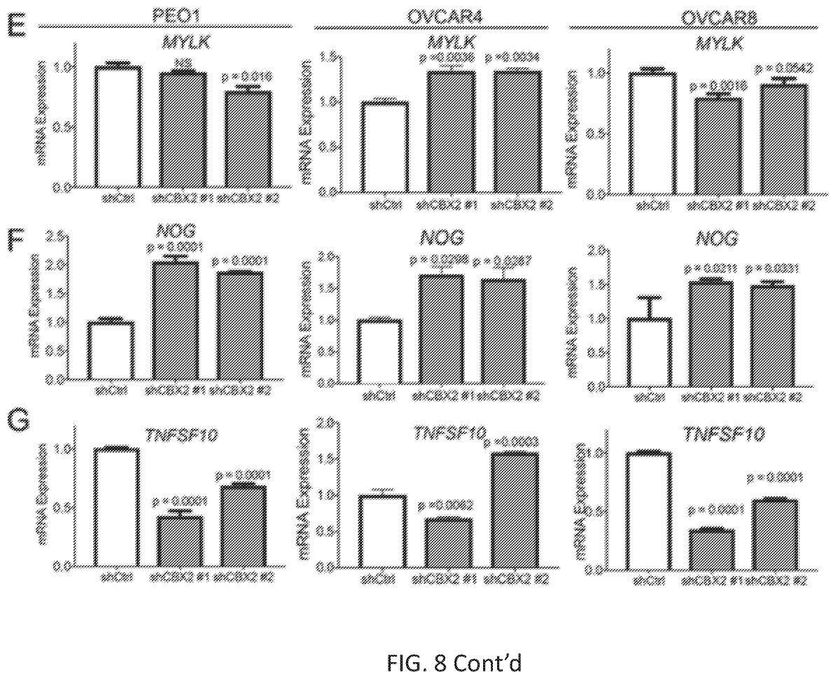

Chemoresistance and dissemination of ovarian cancer cells are associated with changes in apoptosis, epithelial to mesenchymal transition (EMT), and autophagy. The PcG is an epigenetic complex that is responsible for transcriptional reprogramming through histone modification thus CBX2 could regulate a variety of genes. Utilizing HGSOC TOGA data we generated a list of potential CBX2 target genes through examination of mRNA correlations (Spearman r>0.15, 5838 genes). Utilizing published gene sets for EMT, autophagy, stemness, and apoptosis we cross-referenced the CBX2-associated genes (Table 4). CBX2-associated genes accounted for 18.8-28.4% of genes in the respective pathways (FIG. 8 Panel A). We selected a gene from each pathway and in OVCAR4, OVCAR8, and PEO1 observed that the genes were differentially regulated in adherent vs. suspension culture conditions. For instance, in two of the three cell lines an autophagy-related gene, MYLK, and EMT-related gene, NOG, were significantly differentially regulated in suspension (FIG. 8 Panels B, C). Furthermore, we examined an apoptosis-related gene, TNFSF10, and observed that it was upregulated in all three HGSOC cells lines when grown in suspension (FIG. 8 Panel D). Subsequently, CBX2 knockdown cells grown in suspension differentially regulate MYLK, NOG, and TNFSF10 (FIG. 8 Panels E-G). The differential expression observed correlated with the level of CBX2 knockdown (FIG. 2 Panel C and FIG. 3 Panels B-D). These findings suggest that in the context of anoikis CBX2 regulates several pathways, including autophagy, apoptosis, and EMT.

The panels of FIG. 8 are as follows. Utilizing the TOGA (HGSOC, Nature, 2011) dataset, CBX2 expression was correlated to mRNA expression of all genes and 5838 genes (CBX2-associated genes) were identified to have a Spearman correlation of greater than r=0.15. Panel A The 5838 genes were cross-referenced with published gene sets for “Apoptosis”, “Autophagy”, and “Epithelial to Mesenchymal Transition (EMT).” Percentage indicates overlap with gene lists. PEO1, OVCAR4, and OVCAR8 cells were grown in adherent (Adh) or suspension (Sus) for 7 days, RNA was extracted, and used for RT-qPCR against MYLK (Panel B), NOG (Panel C), and TNFSF10 (Panel D). Statistical test=two-sided t-test. shControl and shCBX2 #1 and 2 PEO1, OVCAR4, and OVCAR8 cells were grown in suspension, RNA was extracted, and used for RT-qPCR against MYLK (Panel E), NOG (Panel F), and TNFSF10 (Panel G). Statistical test=ANOVA. Error bars=S.E.M.

### Example 6—Forced Growth in Suspension and Increased CBX2 Leads to a Stem-Like Phenotype

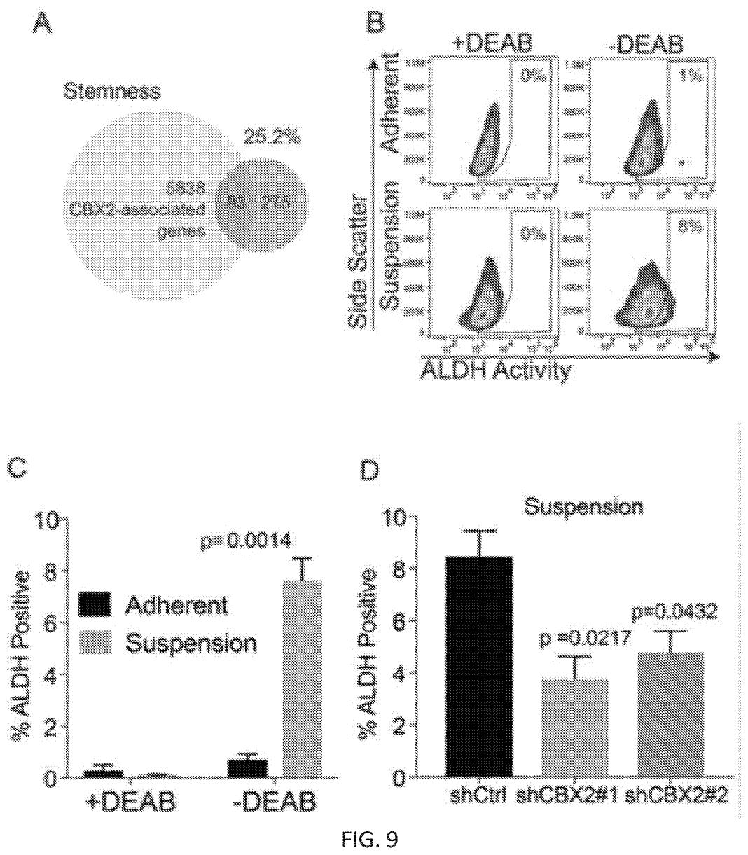

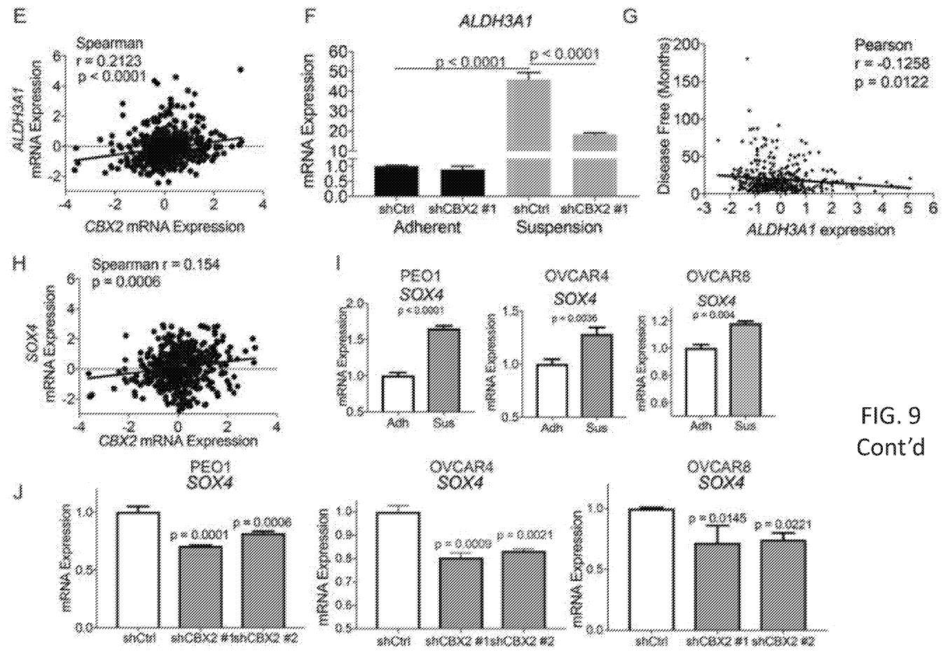

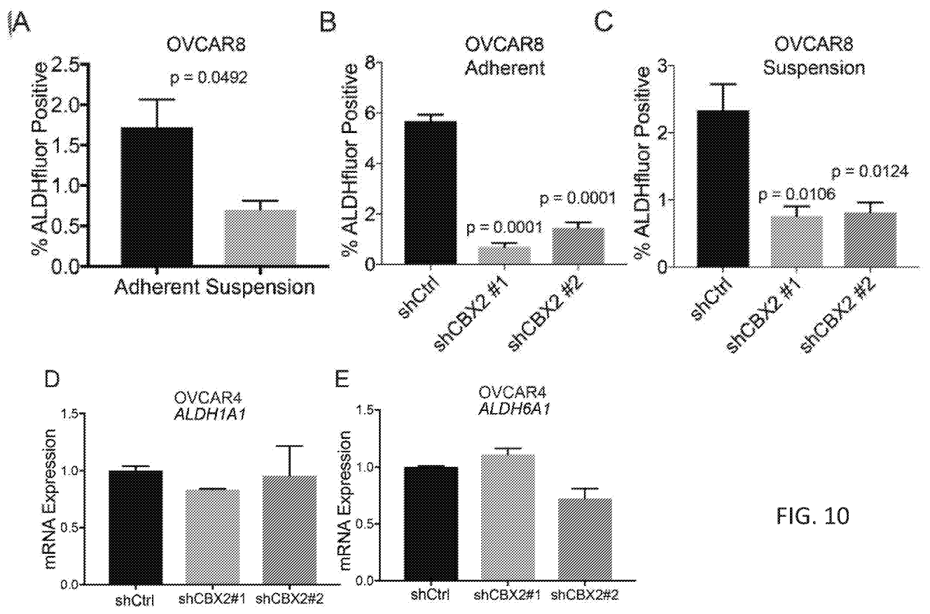

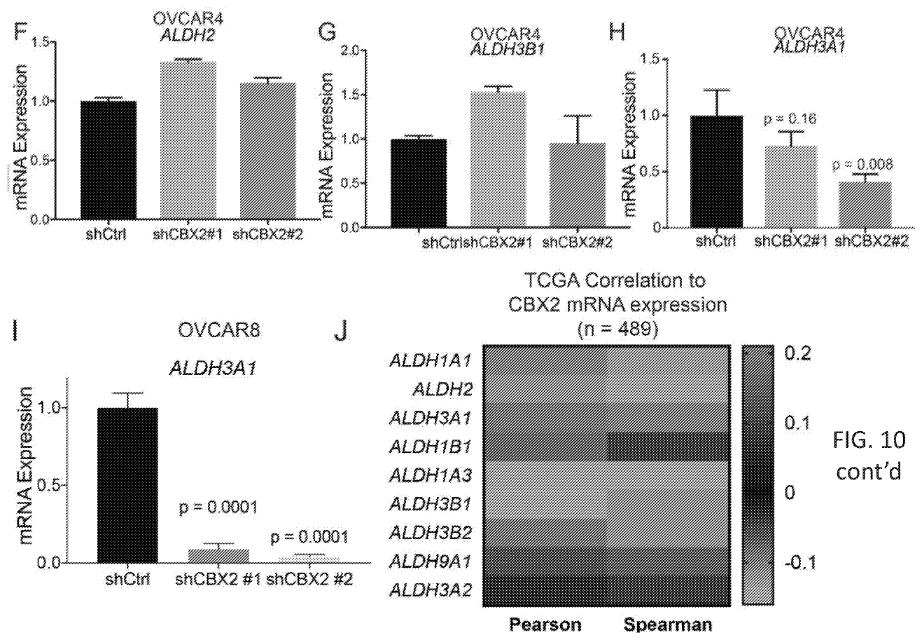

Several reports have demonstrated cells that survive in anchorage-independent conditions possess stem-like characteristics. We hypothesize that the polycomb repressor complex could be involved by inhibiting cellular differentiation and thus maintaining stemness. Applicants examined the CBX2-associated genes from TOGA against a published stemness gene set. We observed 25.2% of stemness-related genes overlapped with CBX2 genes. We next evaluated stemness by measuring aldehyde dehydrogenase (ALDH) activity both in the setting of suspension growth and CBX2 knockdown. We first determined whether placing OVCAR4 and OVCAR8 cells in suspension increased ALDH activity. We observed that in OVCAR4 cells ALDH activity was significantly increased in cells grown in suspension for 7 days (FIG. 9 Panels B and C). In contrast, we did not observe an increase in ALDH activity in OVCAR8 cells grown in suspension for 7 days (FIG. 10 Panel A).

The panels of FIG. 10 are as follows: Panel A: OVCAR8 cells were grown in adherent and suspension conditions for 7 days and utilized for ALDHfluor assay. Percentage of ALDH positive cell graphed. Statistical test=t-test. Panel B: shControl (shCtrl), shCBX2 #1 and #2 OVCAR8 cells were grown in adherent condition for 7 days and utilized for ALDHfluor assay. Percentage of ALDH positive cell graphed. Statistical test=ANOVA. Panel C: Same as B, but examined ALDH positive OVCAR8 cells grown in suspension for 7 days. Statistical test=ANOVA. Panel D: RT-qPCR for ALDH1A1 in OVCAR4 cells transduced with shControl (shCtrl) or shCBX2 #1 and #2. RNA was collected from adherent cells and used for RT-qPCR against CBX2. Panel E: Same as D, but RT-qPCR for ALDH6A1. Panel F: Same as D, but RT-qPCR for ALDH2. G: Same as D, but RT-qPCR for ALDH3B1. Panel H: Same as D, but RT-qPCR for ALDH3A1. Statistical test=ANOVA. I: Same as D, but examined OVCAR cells and RT-qPCR for ALDH3A1. Statistical test=ANOVA. Note: All RT-qPCR 18s was utilized as an internal control. Panel J: TOGA (Nature, 2011, n=489) analysis of HGSOC examining correlation between CBX2 expression and indicated ALDHs. Pearson and Spearman correlations (r values) shown in heatmap. Red=positive correlations and Green=negative correlations.

Next, we evaluated whether CBX2 knockdown impacted suspension-induced ALDH activity, we found that culturing OVCAR4 and OVCAR8 shCBX2 cells in suspension significantly inhibited ALDH activity compared to shControl cells (FIG. 9 Panel D and FIG. 10 Panel B-C). These results highlight that the loss of CBX2 regulates ALDH activity in OVCAR4 and OVCAR8 cell lines. The polycomb repressor complex 2 regulates ALDH1A1 expression, so we sought to determine whether this decrease in ALDH activity correlated with the expression of a specific ALDH gene. We examined the expression of ALHD1A1, ALDH2, ALDH3A1, ALDH3B1, and ALDH6A1 genes in CBX2 knockdown cells (FIG. 10 Panel D-H). ALDH1A1 was not significantly changed in shCBX2 cells. However, in OVCAR4 and OVCAR8 cells, shCBX2 knockdown cells ALDH3A1 expression was significantly decreased (FIG. 10 Panel H-I). Similar to ALDH1A1, ALDH3A1 has previously been associated with stemness. Consistently, the examination of HGSOC samples in the TCGA revealed CBX2 expression correlated with ALDH genes expression with the ALDH3A1 gene having the only significant positive correlation (FIG. 10 Panel J and Table 5). Further analysis of TCGA data comparing CBX2 and ALDH3A1 expression found a significant positive correlation; suggesting CBX2 promotes increased ALDH3A1 expression (FIG. 9 Panel E). In suspension, ALDH3A1 was significantly upregulated compared to adherent cells and knocking down CBX2 significantly abrogated ALDH3A1 expression (FIG. 9 Panel F and FIG. 10 Panels H-I). Furthermore, ALDH3A1 expression significantly correlated with disease-free survival (FIG. 8 Panel G, Pearson's r=−0.1258, p=0.0122). Referring to the TCGA data comparing CBX2 expression we also identified a significant correlation to a potent stem cell-associated transcription factor, SOX4 (FIG. 9 Panel H). In adherent vs. suspension settings SOX4 was significantly upregulated in OVCAR4, OVCAR8, and PEO1 cells grown in suspension (FIG. 9 Panel I). Consequentially, CBX2 knockdown promoted a significant decrease in SOX4 expression (FIG. 9 Panel J). Taken together, these data strongly suggest that HGSOC cells grown in suspension are more stem-like and CBX2 could be promoting stemness through ALDH3A1 and SOX4 regulation.

TABLE 5

| | Pearson | Spearman |
|---|---|---|
| Gene | Correlation (r) | Correlation (r) |
| ALDH1A1 | −0.1 | −0.13 |
| ALDH2 | −0.12 | −0.14 |
| ALDH3A1 | 0.19 | 0.21 |
| ALDH3B1 | −0.08 | −0.12 |
| ALDH1A3 | −0.14 | −0.15 |
| ALDH3B1 | −0.16 | −0.12 |
| ALDH3B2 | −0.09 | −0.12 |
| ALDH9A1 | −0.06 | −0.07 |
| ALDH3A2 | −0.03 | −0.04 |

TCGA Data (n = 489, Nature, 2011)

The panels of FIG. 9 are as follows. Panel A CBX2-associated genes were cross-referenced with a gene set for sternness. Percentage indicates overlap of sternness gene set with CBX2-associated genes. Panel B OVCAR4 cells grown in adherent and suspended settings for 7 days. Aldefluor assay and flow cytometry were utilized to determine the percentage of cells that were positive for aldehyde dehydrogenase (ALDH), a marker of sternness. Diethylaminobenzaldehyde (DEAB), a potent ALDH inhibitor, prevented the increase in ALDH activity and served as negative control (left). Panel C As above, OVCAR4 cells grown in adherent and suspended settings for 7 days. Bar graph compares the percentage of cells ALDH positive control (+DEAB) to cells without DEAB (experimental) in adherent and suspended settings. Statistical test=two-sided t-test. F Test p=0.1136. Panel D OVCAR4 cells with shControl, shCBX2#1 and #2 cultured in suspension over 7 days. Aldefluor assay and flow cytometry again utilized to determine the percentage of cells ALDH positive. Statistical test=two-sided t-test. F-test p=0.83 Panel E Utilizing the TOGA (HGSOC, Nature, 2011, n=489) dataset, a scatter plot CBX2 expression was correlated to ALDH3A1 expression. Spearman correlation r=0.2123 and p-value <0.0001. Panel F RT-qPCR of ALDH3A1 in OVCAR4 cells cultured in adherent and suspension conditions with CBX2 knockdown (shCBX2 #1). Statistical test=ANOVA. Panel G Scatter plot of ALDH3A1 expression (x-axis, Z-score) compared to disease-free survival (y-axis, months). Pearson's correlation r=−0.1258 and p-value=0.0122. Panel H Utilizing the TOGA (HGSOC, Nature, 2011, n=489) dataset, a scatter plot CBX2 expression was correlated to SOX4 expression. Spearman correlation r=0.154 and p-value=0.0006. Panel I PEO1, OVCAR4, and OVCAR8 cells grown in adherent (Adh) or suspension (Sus) settings for 7 days. RNA was extracted, and used for RT-qPCR against SOX4. Statistical test=two-sided t-test. Experiment performed in technical triplicates and biological duplicate. Panel J shControl and shCBX2 #1 and #2 PEO1, OVCAR4, and OVCAR8 were cultured in suspension conditions, RNA was extracted and used for RT-qPCR against SOX4. Experiment performed in technical triplicates and biological duplicate. Statistical test=ANOVA. Error bars=S.E.M.

### Materials and Methods

### 1. Cell Culture

OVCAR4, PEO1, and OVCAR8 human high grade serous ovarian cancer cell lines were authenticated using small tandem repeat (STR) analysis (The University of Arizona Genetics Core) and routinely tested for mycoplasma with MycoLookOut (Sigma, St. Louis, Mo.). OVCAR8 and OVCAR4 cells were obtained from the Gynecologic Tumor and Fluid Bank (University of Colorado, Aurora, Colo.). PEO1 purchased from American Type Culture Collection. Cells were cultured in RPMI-1640 medium supplemented with 1% penicillin—streptomycin and 10% fetal bovine serum. The cell lines were maintained in 5% CO2 at 37° C.

### 2. Bioinformatic Database Analysis

Gene Expression Omnibus (GEO), hosted by the National Center for Biotechnology Information (NCBI), was queried for relevant databases. GSE18521, GSE10971, GSE1926, and GSE73064 were examined for relative CBX2 expression. The Cancer Genome Atlas (TCGA) Ovarian Serous Cystadenocarcinoma (Nature 2011) database was accessed via the cBIOPortal (www.cbioportal.org/) to evaluate the role of CBX2 in HGSOC disease-free and overall survival. Note: patient numbers for overall survival (n=485) and disease-free survival (n=396) differ due to the availability of data with TCGA. The database was queried for HGSOC identifying a total of 557 tumors with mRNA expression data and CBX2 upregulation was defined as CBX2 mRNA expression >1.5 standard deviation. RPPA data was assessed from the HGSOC Provisional dataset via the cBIOPortal.

### 3. Adherent and Suspension Environments

The adherent environment was created using standard tissue culture dishes. For suspension, tissue culture dishes were covered with 6 mg/ml poly-2-hydroxyethyl methacrylate (Poly-HEMA, Sigma) in 95% ethanol. The plates were incubated under sterile conditions to allow ethanol evaporation, followed by 30 min of ultraviolet light for sterilization. OVCAR4, PEO1, and OVCAR8 cells were cultured in each of these environments for 7 days.

### 4. Cisplatin Dose-Response

PEO1 and OVCAR4 cells were plated in 96-well plates in both adherent (4000 cells per well) and suspension (6000 cells per well) environments, as described above. These cells were treated over 24 h with increasing concentrations of Cisplatin (0.5-100 μM). 1640 RPMI media and media with 0.9% NaCl were used for control and vehicle control, respectively. Cell viability was assessed using the 3-(4,5-dimethylthiazol-2-yl)-5-(3-carboxymethoxyphenyl)-2-(4-sulfophenyl)-2H-tetrazolium (MTT) assay (Promega, Madison, Wis.). Means of at least five wells are reported and experiments were independently repeated in triplicate. Representative dose-response curves are shown.

### 5. Quantitative Reverse Transcription Polymerase Chain Reaction (RT-qPCR)

RNA was isolated using RNAeasy Plus Mini Kit (Qiagen, Hilden, Germany) according to the manufacturer's protocol. NanoDrop spectrophotometry was performed to confirm the concentration of extracted RNA. RT-qPCR was performed using the Luna Universal One-step RT-qPCR kit (New England BioLabs, Ipswich, Mass.) on a BioRad CFX96 or Applied Biosystems QuantStudio 6 Flex thermocycler using primers for specific target transcripts; 18s rRNA was examined as a housekeeping gene (Table 6).

TABLE 6

| | | | SEQ |
|---|---|---|---|
| | | | ID |
| Gene | Direction | Sequence (5′-3′) | NO: |
| CBX2 | Forward | CGGCTGGTCCTCCAAACATAA | 1 |
| CBX2 | Reverse | CAGAACCGGAAGAGAGGCAA | 2 |
| 18sRNA | Forward | AACTTTCGATGGTAGTCGCCG | 3 |
| 18sRNA | Reverse | CCTTGGATGTGGTAGCCGTTT | 4 |
| ALDH3B1 | Forward | TACGCCTTCTCCAACAGCAG | 5 |
| ALDH3B1 | Reverse | GTCATGTGCATGAAGCCGTC | 6 |
| ALDH2 | Forward | GGTGTGGTCAACATTGTGCC | 7 |
| ALDH2 | Reverse | ATTACGCGGCCAATCTCAGT | 8 |
| ALDH3A1 | Forward | AAGAGGAGATCTTCGGGCCT | 9 |
| ALDH3A1 | Reverse | TAATCACCTTGTCGTTGCTGG | 10 |
| | | A | |
| MYLK | Forward | TCAACAGGGTCACCAACCAG | 11 |
| MYLK | Reverse | GGGGTCTGGGTATCCTTCAAT | 12 |
| NOG | Forward | TGGTGGACCTCATCGAACAC | 13 |
| NOG | Reverse | ATGAAGCCTGGGTCGTAGTG | 14 |
| TNFSF10 | Forward | TGCGTGCTGATCGTGATCTT | 15 |
| TNFSF10 | Reverse | CATTCTTGGAGTCTTTCTAAC | 16 |
| | | GAGC | |
| SOX4 | Forward | CCCAGCAAGAAGGCGAGTTA | 17 |
| SOX4 | Reverse | CATCGGCCAAATTCGTCACC | 18 |

6. shRNA Knockdown

CBX2-specific shRNA were obtained from the University of Colorado Functional Genomics Facility (CBX2 #1: TRCN 0000020327 and CBX2 #2: TRCN 0000020328). An empty pLKO.1-puro was utilized as shControl (shCtrl). Plasmid isolation was performed using Plasmid Midi-Prep Kit (Qiagen). Twenty-four hours after seeding, cells were transfected with a total of 12 μg of DNA, including lentiviral packaging plasmids and the shRNA, in addition to 36 μg of polyethyenimine (PEI), for a 1:3 ratio of DNA to PEI. Cells were incubated overnight and transitioned to Dulbecco's Modified Eagle Media (DMEM) the following morning. Forty-eight hours after medium change, lentivirus was harvested. PEO1, OVCAR4, and OVCAR8 cells were seeded into six-well plates. When cells reached 80% confluence, they were transduced with lentivirus encoding CBX2-specific shRNAs or an shRNA control. A control well was maintained without virus to confirm puromycin selection. A 48-h puromycin selection was performed immediately following transduction. After medium change, cells were allowed to recover and then subjected to functional assays.

### 7. Immunoblot

Cell lysis was performed using radioimmunoprecipitation assay (RIPA) buffer (150 mM sodium chloride, Triton X-100, 0.5% sodium deoxycholate, 0.1% SDS [sodium dodecyl sulfate], 50 mM Tris, pH 8.0) supplemented with complete EDTA-free protease inhibitor cocktail (Roche), as well as NaF and NaV. Protein was quantified using bicinchoninic acid (BCA) protein assay (Thermo Fisher Scientific, Waltham, Mass.) and spectrophotometry. An 8% SDS polyacrylamide gel resolving gel was created with a 4% stacking gel. Twenty to thirty micrograms of total protein were loaded per well. Proteins were transferred to PVDF membrane using a Bio-Rad TransBlot Turbo. Western Blot analysis was performed using a rabbit primary antibody specific to CBX2 (Thermo Fisher Scientific, Cat # PA5-30996, 1:1000) and a mouse primary antibody against actin (Abcam, Cat # ab6276). CBX2 previously validated in Clermont, P. L. et al. Genotranscriptomic meta-analysis of the Polycomb gene CBX2 in human cancers: initial evidence of an oncogenic role. Br. J. Cancer 111, 1663-1672 (2014). Primary antibody incubation was performed overnight at 4° C. Secondary goat anti-rabbit green (LI-COR Biosciences, Lincoln, Nebr., Cat #926-32211) and goat anti-mouse red (LI-COR, Cat #926-68070) antibodies were applied the following morning for 1 h at room temperature. Bands were visualized using the LI-COR Odyssey Imaging System.

### 8. Proliferation Assays

For the Gaussia luciferase (gLuc) assay, the BioLux Gaussia Luciferase Assay kit (New England BioLabs) was utilized. OVCAR4 and PEO1 cells were grown in a 96-well plate, starting with 2000 cells per well. Media were collected every 24 h and stored in at −20°. For the assay and luminometer readings, the media were thawed and placed in a new 96-well plate. The assay was performed following the manufacturer's protocol and the relative light units were obtained using luminometry (GloMax) and charted with Prism software. Colony formation assays were performed in parallel using crystal violet staining. Briefly, cells were fixed (10% methanol/10% acetic acid/PBS), stained with crystal violet (0.4%) and washed with de-ionized water. Crystal violet was dissolved and absorbance was measured using a spectrophotometer (SpectraMaxM2e, Molecular Devices, San Jose, Calif.) at 590 nm and SoftMaxPro software. For the spheroid assay, 4000 cells were plated from a single cell suspension onto growth factor reduced Matrigel (Corning, Corning, N.Y.) and allowed to incubate for 12 days. Microscopic images were obtained and the diameter of each spheroid was measured in ImageJ (NIH). At least 50 spheroids were measured for each cell type and the diameters were averaged and graphed using Prism software.

### 9. Aldefluor Assays

Stemness was evaluated using the Aldefluor Kit (Cat #01700, Stemcell Technologies, Vancouver, Canada). OVCAR4 and OVCAR8 cells grown in adherent and suspension settings for 7 days were collected and prepared following manufacturer's protocol. An exception to the manufacturer's protocol was the reduction of Aldefluor reagent by 50%, using 2.5 μl per 1 ml of cell suspension. Flow cytometry was performed on a Gallios 561 Cytometer (BD Biosciences) with analysis at 488 nm

### 10. Gynecologic Tissue and Fluid Bank (GTFB)

The University of Colorado has an Institutional Review Board approved protocol (COMIRB #07-935) in place to collect tissue from gynecologic patients with both malignant and benign disease processes. All participants are counseled regarding the potential uses of their tissue and sign a consent form approved by the Colorado Multiple Institutional Review Board. The tissues are processed, aliquoted, and stored at −80° C.

### 11. Immunohistochemistry

Patient-derived xenograft tissue samples of HGSOC were fixed in formalin and embedded in paraffin. The University of Colorado Cancer Center Histology Core performed serial sectioning of the tissue at 5 micron thickness. For histopathologic examination, sections were de-paraffinized using xylene and hydrated in graded alcohol solutions. Antigen retrieval was performed using citrate buffer (pH 6.0) and boiling in pressurized steamer to 110° C. for 30 min. Endogenous peroxidase activity was quenched with 3% hydrogen peroxide in methanol for 20 min, followed by washing in TBS. A hydrophobic barrier was drawn around each section and tissues were blocked in 1% BSA in TBS for 30 min. TMA slides were single stained. Rabbit anti-CBX2 (Thermo Scientific, Cat # PA5-30996) was diluted to 1:50 in 1% BSA in TBS, applied to all sections, and incubated overnight at 4° C. Rabbit anti-PAX8 (Proteintech, Cat #10336-1-AP) was diluted to 1:200 in 1% BSA in TBS, applied to all sections, and incubated overnight at 4° C. An isotype control (Rabbit IgG) was incubated in parallel. The secondary antibody, anti-rabbit Dako Envision+System HRP Labeled Polymer (Dako Ref#K4003) was applied to the sections and allowed to incubate for 60 min at room temperature. Slides were subsequently washed with TBS and developed under the microscope using Liquid 3,3′-diaminobenzidine tetrahydrochloride (DAB)+Substrate Chromagen System (Agilent, Santa Clara, Calif.; Ref#K3468). Slides were counterstained with hematoxylin

### 12. Tissue Microarray

A previously constructed TMA comprised of matched primary, lymph node, and peritoneal metastases samples in duplicate from 24 patients with high grade serous carcinoma treated at the University of Colorado (COMIRB #14-0427), was stained with the CBX2 and PAX8 antibodies. With the aid of the University of Colorado Histology Core, the stained slides were scanned using Aperio imaging technology and annotated to highlight tumor based on PAX8 staining using ImageScope software. The TMA was then analyzed and scored by the University of Colorado Histology Core. Subsequently, two board-certified pathologists (M.D.P. and A.A.B.) manually reviewed and scored the CBX2 stain. Each sample was given a score for intensity (0, 1+, 2+, 3+) and percentage of cells staining (continuous variable). Only distinct nuclear staining of tumor cells was considered positive. From this data, H-scores were generated.

### 13. Apoptosis Assays

Following 72 h of growth in adherent and suspended states, OVCAR4 and PEO1 cells were harvested and washed in PBS. Alexa 488 Conjugated AnnexinV and Propidium Iodide (PI) (Thermo Fisher Scientific) staining were performed following manufacturer's protocol.

### 14. Statistical Analysis

Prism Graph Pad Prism software (v7) was utilized to generate graphs. Statistical tests include unpaired two-sided t-tests (comparing two groups), Log-rank (survival) or ANOVA (comparing greater than two groups) unless noted. A significance threshold was set at p<0.05, which was used for sample size determination. All experiments were performed in technical triplicates and biological triplicates unless noted. FloJo software (BD Biosciences, San Jose, Calif.) was used for analyzing flow cytometry data.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1-3. (canceled)

4. A method of treating HGSOC in a patient, the method comprising:
administering to the patient a therapeutically effective amount of a compound that inhibits and/or downregulates CBX2;
contacting the compound with at least one cell in a tissue selected from ovarian, uterine, peritoneal, or fallopian tissue;
downregulating CBX2 in that cell, thereby treating HGSOC in the patient.

5. The method of claim 4, wherein the compound comprises an antibody, siRNA, ribozyme, antisense, aptamer, peptidomimetic, small molecule, or any combinations thereof.

6. A method of determining and selecting a treatment for a patient with, or at risk of developing a cancer, the method comprising:
obtaining a first sample from the patient comprising one or more cancerous or pre-cancerous cells;
obtaining a second sample from the patient that is similar to the first sample but does not comprise cancerous or pre-cancerous cells;
processing the first sample and the second sample to analyze at least one biomarker related to CBX2;
quantifying the amount of biomarker in the first sample and the second sample, wherein if the amount of CBX2-related biomarker in the first sample is greater than the amount of CBX2-related biomarker in the second sample, the patient is identified as having aggressive or chemoresistant cancer; and
selecting a chemotherapy for the patient that is not a platinum-based chemotherapeutic agent if the patient is identified as having aggressive or chemoresistant cancer, or a chemotherapy that is a platinum-based chemotherapeutic agent if the patient is not identified as having aggressive or chemoresistant cancer.

7. The method of claim 6, wherein the first sample is derived from ovarian, uterine, peritoneal, or fallopian tissue.

8. (canceled)

9. The method of claim 7, wherein the first sample includes one or more HGSOC cells.

10. The method of claim 9, wherein the biomarker comprises CBX2 protein or fragment thereof.

11. The method of claim 10, further comprising obtaining a second biomarker from each sample, wherein the second biomarker is not CBX2.

12. The method of claim 11, wherein the amount of biomarker is quantified by immunochemistry.

13. The method of claim 11, wherein the amount of biomarker is quantified by mass spectrometry.

14. The method of claim 9, wherein the biomarker is at least one nucleic acid.

15. The method of claim 14, wherein the biomarker is an mRNA sequence from the CBX2 gene.

16. The method of claim 6, wherein the platinum-based chemotherapeutic agent is cisplatin.

17-36. (canceled)

37. A method of inhibiting or reducing proliferation in a cancer cell, the method comprising:
contacting the cell with a compound or molecule that inhibits CBX2 expression;
allowing the compound to reduce the amount of CBX2 protein in the cell; thereby
reducing or inhibiting proliferation of the cell compared to a control cell that is not contacted with the compound.

38. The method of claim 37, wherein the cell is in-vitro.

39. The method of claim 37, wherein the cell is in-vivo.

40. (canceled)

41. The method of claim 37, wherein the cell is a human cell.

42. The method of claim 37, wherein the compound is a nucleic acid.

43. The method of claim 42, wherein the compound is a short hairpin ribonucleic acid.

44. The method of claim 37, wherein the compound comprises two or more amino acids.

45. The method of claim 37, further comprising a step of contacting the cell with one or more chemotherapeutic agents before the reducing step.


SEQUENCE LISTING (20 sequences)

SEQ ID NO: 1 - DNA, 21 bp, Homo sapien
cggctggtcctccaaacataa

SEQ ID NO: 2 - DNA, 20 bp, Homo sapien
cagaaccggaagagaggcaa

SEQ ID NO: 3 - DNA, 21 bp, Homo sapien
aactttcgatggtagtcgccg

SEQ ID NO: 4 - DNA, 21 bp, Homo sapien
ccttggatgtggtagccgttt

SEQ ID NO: 5 - DNA, 20 bp, Homo sapien
tacgccttctccaacagcag

SEQ ID NO: 6 - DNA, 20 bp, Homo sapien
gtcatgtgcatgaagccgtc

SEQ ID NO: 7 - DNA, 20 bp, Homo sapien
ggtgtggtcaacattgtgcc

SEQ ID NO: 8 - DNA, 20 bp, Homo sapien
attacgcggccaatctcagt

SEQ ID NO: 9 - DNA, 20 bp, Homo sapien
aagaggagatcttcgggcct

SEQ ID NO: 10 - DNA, 22 bp, Homo sapien
taatcaccttgtcgttgctgga

SEQ ID NO: 11 - DNA, 20 bp, Homo sapien
tcaacagggtcaccaaccag

SEQ ID NO: 12 - DNA, 21 bp, Homo sapien
ggggtctgggtatccttcaat

SEQ ID NO: 13 - DNA, 20 bp, Homo sapien
tggtggacctcatcgaacac

SEQ ID NO: 14 - DNA, 20 bp, Homo sapien
atgaagcctgggtcgtagtg

SEQ ID NO: 15 - DNA, 20 bp, Homo sapien
tgcgtgctgatcgtgatctt

SEQ ID NO: 16 - DNA, 25 bp, Homo sapien
cattcttggagtctttctaacgagc

SEQ ID NO: 17 - DNA, 20 bp, Homo sapien
cccagcaagaaggcgagtta

SEQ ID NO: 18 - DNA, 20 bp, Homo sapien
catcggccaaattcgtcacc

SEQ ID NO: 19 - DNA, 21 bp, Homo sapien
gccaaggaagctcactgccat

SEQ ID NO: 20 - DNA, 21 bp, Homo sapien
acggaaaggaacaggaagcat